(19) United States
(12) Patent Application Publication
(10) Pub. No.: US 2025/0143580 A1
(43) Pub. Date: May 8, 2025

(54) ORAL SCANNER SYSTEM

(71) Applicant: Braun GmbH, Kronberg (DE)

(72) Inventors: Ingo VETTER, Karben (DE); Reiner ENGELMOHR, Egelsbach (DE); Bettina ROWLANDS, Bad Homburg (DE); Faiz Feisal SHERMAN, Mason, OH (US); Pei LI, Eschborn (DE); Xinru CUI, Beijing (CN)

(21) Appl. No.: 19/015,029

(22) Filed: Jan. 9, 2025

Related U.S. Application Data
(63) Continuation of application No. PCT/CN2023/102654, filed on Jun. 27, 2023.

(30) Foreign Application Priority Data
Jul. 4, 2022 (WO) PCT/CN2022/103552
Jul. 4, 2022 (WO) PCT/CN2022/103580
Jul. 4, 2022 (WO) PCT/CN2022/103583
Jul. 4, 2022 (WO) PCT/CN2022/103603
Jul. 4, 2022 (WO) PCT/CN2022/103648
Jul. 4, 2022 (WO) PCT/CN2022/103673

(51) Int. Cl. A61B 5/00

(52) U.S. Cl. CPC A61B 5/0088; A61B 5/7425; A61B 2562/0219; A61B 2562/028

(57) ABSTRACT

The present application is concerned with an oral scanner system having an oral scanner comprising an oral health sensor structured for acquiring oral health sensor data from at least a portion of a dentition of a subject during a scanning procedure relating to at least one oral health condition, and a position detector structured for acquiring position sensor data during the scanning procedure, a processor structured to receive the oral health sensor data and the position sensor data, to process the position sensor data to determine a discrete position or location from at least two discrete positions or locations of the at least portion of the dentition at which the oral scanner is currently performing the scanning procedure or has been performing the scanning procedure at a given time instant, to assign the oral health sensor data to one discrete position of the at least two discrete positions to create discretely position-resolved oral health sensor data, to compare the discretely position-resolved oral health sensor data with historic discretely position-resolved oral health sensor data, being stored in a memory of the oral scanner system and having been determined in at least one previous scanning procedure, and to generate comparison data and to control a display unit to provide a visualization of the discretely position-resolved or location-resolved oral health sensor data at least after the scanning procedure is complete and/or to provide a visualization of comparison data.

### FIELD OF THE INVENTION

The present disclosure is concerned with an oral scanner system that comprises an oral scanner having an oral health sensor and a processor structured and arranged for the determination of oral health data relating to at least one oral health condition.

### BACKGROUND OF THE INVENTION

Various professional and home use oral scanner systems are known to the skilled person.

There is a general interest, specifically for home-use oral scanner systems, to provide an oral scanner system that enables an improved ease of usage or improved guidance through an oral scanning procedure and/or a more versatile and ideally intuitively understandable communication of oral health data relating to at least one oral health condition in the context of a continuous oral care treatment. In accordance with some aspects, there is a desire to provide at least an alternate oral scanner system.

### SUMMARY OF THE INVENTION

In accordance with at least one aspect, an oral scanner system is provided comprising an oral scanner comprising an oral health sensor structured and/or arranged for acquiring oral health sensor data from at least a portion of a dentition of a subject during a scanning procedure relating to at least one oral health condition, and a position detector structured and/or arranged for acquiring position sensor data during the scanning procedure, a processor structured and/or arranged to receive the oral health sensor data and the position sensor data, to process the position sensor data to determine a discrete position or location from at least two discrete positions or locations of the at least portion of the dentition at which the oral scanner is currently performing the scanning procedure or has been performing the scanning procedure at a given time instant, to assign the oral health sensor data and/or oral health data determined by the processor based on the oral health sensor data to one discrete position or location of the at least two discrete positions or locations to create discretely position-resolved or location-resolved oral health sensor data and/or oral health data, to compare the discretely position-resolved or location-resolved oral health sensor data and/or oral health data with historic discretely position-resolved or location-resolved oral health sensor data and/or historic oral health data, respectively, being stored in a memory of the oral scanner system and having been determined in at least one previous scanning procedure, and to generate comparison data, preferably discretely position-resolved or location-resolved comparison data, and to control a display unit to provide a visualization of the discretely position-resolved or location-resolved oral health sensor data and/or the oral health data at least after the scanning procedure is complete and/or to provide a visualization of comparison data, preferably the discretely position-resolved or location-resolved comparison data.

The above aspects that are in more detailed discussed in the following assist the user in the usage of the oral scanner system by means of a continued and/or guided human-machine interaction. The feedback in comparison to the historic data support a more optimized usage of the oral scanner system. In addition to the oral scanner system as discussed herein, a method of scanning at least a portion of the oral cavity using an oral scanner system is also considered.

### BRIEF DESCRIPTION OF THE DRAWINGS

The present disclosure will be further elucidated by a detailed description of example embodiments and with reference to figures. In the figures

FIG. 1 is a schematic depiction of an example oral scanner system comprising an oral scanner and a processor disposed in the oral scanner;

FIG. 2 is a schematic depiction of an example oral scanner system comprising an oral scanner and a separate device realizing or comprising the processor;

FIG. 3 is a schematic depiction of the basic components enabling a position or location determination for an oral scanner, where the outcome of the position or location determination is the position or location in the oral cavity at which a head of the oral scanner currently performs a scanning procedure;

FIG. 4 is a schematic depiction of another example oral scanner system comprising an oral scanner and an oral care device and further optional components such as a charger and where the oral scanner system may be structured and/or arranged to communicate data between its various components and to a remote computing instance;

FIG. 5 is a depiction of an example feedback screen as may be visualized on a display being part of an oral scanner system, where the feedback screen comprises the visualization of a live or conserve image taken in a scanning procedure and an abstract visualization of a dentition onto which scanning progress data and oral health data are overlaid in a live manner;

FIG. 6 is a depiction of another feedback screen as may be visualized on a display being part of the oral scanner system, where the feedback screen shows a summary of oral health data overlaid on an abstract depiction of a dentition and further a trend of a temporal development of an oral health condition is visualized in the center of the screen; and

FIG. 7 is a depiction of another feedback screen as may be visualized on a display being part of the oral scanner system, where various oral health data are visually overlaid onto an abstract depiction of the dentition and further a classification of the related oral health conditions is visually provided.

### DETAILED DESCRIPTION OF THE INVENTION

The following is a general disclosure of example oral scanner systems comprising example oral scanners and example processors and further optional components such as a separate device realizing at least a part of a feedback unit, e.g., comprising a display, and/or an oral care device. The phrase “structured and/or arranged” used in the present disclosure refers to structural and/or computer-implemented features of the respective component and this shall imply that the respective feature or component is not only suited for something but is structurally and/or software-wise arranged to indeed perform as intended in operation. It is here highlighted that the oral scanner in accordance with the present disclosure is understood to be an oral scanner that does not provide any oral care activity as such, in particular does not comprise any oral cleaning elements, i.e., is free from oral cleaning elements or other oral treatment or care elements and does not provide any oral cleaning or oral treatment or oral care. In other words, the present disclosure is concerned with an oral scanner having at least one oral health sensor without any further oral cleaning/treatment/care features. As will be discussed, such a sole oral scanner device may cooperate directly or indirectly with an oral care device which is structured and/or arranged to provide an oral care activity. In such a system, the oral scanner and the oral care device are specialized devices optimized for the individual task and can benefit from information previously recorded by one or the other device, e.g., the oral scanner may scan regions and/or segments of low oral activity by the oral care device and, vice versa, the oral care device may feedback to the user to increase an oral care activity in regions and/or segments where the oral scanner had determined the presence of an oral health issue.

### GENERAL DISCUSSION

The present disclosure is concerned with an oral scanner system that comprises at least an oral scanner and a processor, where the processor may be physically located at or inside of the oral scanner or may be realized as a processor that is separate, i.e., remote from the oral scanner. As will be discussed in more detail further below, the processor may also be realized in a distributed manner. The oral scanner system may specifically comprise at least one separate or remote device that, e.g., realizes at least a part of a feedback unit such as a display. This shall not exclude that, e.g., the oral scanner itself alternatively or additionally comprises a display and/or at least one visual feedback element. A remote display and a remote processor may be arranged together in a separate device, i.e., they may have a joint outer housing. The separate device may be a proprietary or custom-made device, e.g., a charger with a display, or a generally known device such as a computer, a laptop, a notebook, a tablet, a phone like a mobile phone or smartphone, or a smart watch, which may be used to realize a separate display and/or a separate processor. The oral scanner system may alternatively or additionally to the separate device comprise at least one oral care device like a toothbrush, specifically an electric toothbrush that may at least for a limited time period be directly or indirectly coupled with the oral scanner and/or the processor, preferably coupled for the exchange of data such as by wireless communication. The oral scanner and the oral care device may share the same handle and only become as such realized by attaching a respective oral scanner head or an oral care head to the handle. But it may be preferred to have an independent oral care device that has its own handle and that may be arranged to also be used independently from the oral scanner system, i.e., hardware-wise decoupled from the oral scanner system. It may be foreseen that an oral care device may first need to be registered with the oral care system to become a part of the oral care system. The oral scanner system may comprise at least one charger for charging a rechargeable energy storage of the oral scanner and/or of the oral care device and/or of the separate device. The charger(s) may be a wireless charger, such as an inductive charger.

The oral scanner may comprise at least one oral health sensor for acquiring, detecting, measuring or determining and for outputting oral health sensor data relating to at least one oral health condition—where in the following one of the terms “acquiring”, “detecting”, “measuring” or “determining” (or other forms of these verbs or nouns derived from these verbs) is used in connection with an oral health sensor, this shall include the other terms as well. The oral scanner system may comprise at least one position sensor that is structured and/or arranged to provide, i.e., output position sensor data that allows to detect, measure or determine at least one discrete position or location (or: segment) at which the oral scanner currently performs a scanning procedure or has been performing a scanning procedure at a given time instant, where the scanning procedure includes the determination of oral health sensor data. In the present context, the term “discrete” in connection with the position of location within the oral cavity shall indicate that the oral cavity is split into or more discrete regions or segments, e.g., the upper jaw and the lower yaw. Typically, the discrete regions or segments are non-overlapping and essentially completely or in a gap-free manner cover the portion of the oral cavity that is intended for being scanned.

It is mentioned here that the aim of the present proposal is to provide an easy-to-digest information to the user where, e.g., acquired oral health information or scanning procedure progress information or the like is provided in a processed manner per discrete position or location (or: per segment), specifically reduced to a single value or single indicium, i.e., a single percentage value representing the currently or finally achieved scanning procedure progress or an oral health condition or a color indicating the achieved scanning procedure progress or an oral health condition. The present oral scanner system is specifically intended for home use by a layman and thus the improvements and benefits associated with the present proposals are specifically seen for such a home-use by a non-professional user.

The term “sensor” shall be understood to cover sensor types that measure or determine a parameter relevant for an oral health condition based on an external measurement medium such as ambient light impinging onto the sensor or saliva available in the oral cavity being analyzed by the sensor, i.e., sensors comprising a sensor receiver. The term “sensor” shall further cover sensor types comprising a sensor emitter arranged for emitting a measurement medium such as light, i.e., a light emitter, and a sensor receiver such as a light receiver so that the measurement or determination depends at least in part on a non-external measurement medium, which means a measurement medium that is provided by the respective sensor emitter. The oral scanner is structured and/or arranged for performing a scanning procedure in which the oral scanner acquires oral health sensor data from at least a portion of the oral cavity via an oral health sensor, preferably oral health sensor data relevant for determining oral health data relating to the at least one oral health condition. Preferably, the oral health sensor data and/or the therefrom determined oral health data are acquired in a position-resolved or location-resolved manner, i.e., where the respective oral health sensor data and/or oral health data is assigned to position data or location data derived from position sensor data acquired by the position sensor with respect to the same time instant or period of time at or during which the oral health sensor data was acquired.

In the context of the present disclosure, the term “oral health sensor data” refers to the essentially unprocessed data outputted by the oral health sensor during the scanning procedure (e.g., image data if the oral health sensor is a camera or a pH value if the oral health sensor is a pH sensor) and the term “oral health data” refers to processed oral health sensor data (e.g., normalized or absolute area per tooth or per discrete position or location showing plaque or averaged pH value per discrete position or location). It shall be understood that in some instances the oral health sensor data is itself a direct measure for the oral health condition, e.g., the oral health sensor data from a malodor sensor may not need any further processing to allow to determine whether or not the user has a bad odor as the malodor sensor may provide a level of sulfur emissions. The processing of the oral health sensor data may then be considered a classification of the oral health sensor data into one of at least two condition classes, e.g., “no relevant level of malodor” as one condition class and “relevant level of malodor” as another condition class. The classification may then be done by the processor by a comparison with at least one threshold value. More complex classification concepts are discussed below. Of course, a classification as just described can also use the processed oral health sensor data, i.e., the oral health data. E.g., the output from a malodor sensor may be averaged over several measurement instances and then be used for the classification.

The processor is coupled with the oral health sensor and/or with the position sensor to receive at least one sensor datum, preferably a plurality and/or sequence of sensor data, where a single sensor datum may be received in temporal sequence to accumulate to a plurality of temporally spaced sensor data or a plurality of sensor data may be received at each measurement time instant so that this accumulates to a multiplicity of temporally spaced pluralities of sensor data. Sensor data may be transmitted to the processor as sensor signals, e.g., a sensor signal may be a voltage signal as is often the output of a sensor measuring a physical, chemical or material property. The sensor signals may be analog signals or digital signals. The term “datum” or “data” here refers to the information content and “signal” to the physical quantity by which the sensor datum or sensor data is/are transmitted. Where in the present disclosure the term “sensor data” is used, this shall refer to “oral health sensor data” provided by the oral health sensor and to “position sensor data” provided by the position sensor. Where only one of the two types of data is intended to be meant, the respective more limited term will be used. The processor is preferably arranged to process sensor data from the at least one oral health sensor and the at least one position sensor so that at least one position-resolved or location-resolved oral health datum relating to at least one oral health condition is determined. That said, it is clarified that the oral health sensor outputs oral health sensor data that may be processed by the processor to determine oral health data and the position sensor outputs position sensor data that is processed by the processor to determine position data and the processor may further relate or assign oral health data and position data to each other so that position-resolved or location-resolved oral health data results. As mentioned, the oral health sensor data may be assigned to the position data without a need of further processing of the oral health sensor data.

The oral scanner may comprise a scanner head and a scanner handle, which may be detachably connected, even so this shall not exclude that the scanner head and the scanner handle may be non-detachably connected and may form one integral device. The oral scanner may have a housing enveloping a hollow in which components of the oral scanner may be disposed such as an energy source, a controller, a scanner communicator etc. The housing may allow a user to grasp the oral scanner conveniently with a hand. The scanner head may be sized and shaped to be conveniently inserted into the oral cavity. The housing may accommodate at least one user-operable control element such as an on/off button or on/off switch or a selector button or selector switch or other such elements typically expected or found on an oral scanner. The housing may further accommodate at least one feedback element of a feedback unit structured and/or arranged for providing user-perceptible feedback to the user. The feedback unit may comprise one or several feedback elements, e.g., a display provided by a separate device. The at least one feedback element may comprise at least one from the list comprising, in a non-limiting manner, an optical feedback element such as a light emitter or a plurality of light emitters or a display, an acoustic feedback element such as a loudspeaker or a piezo-electric speaker or buzzer, and a tactile or haptic feedback element such as a vibrator or any other type of tactile or haptic feedback generator, e.g., a refreshable braille display.

In embodiments where the oral scanner system comprises a display as an element of a feedback unit, e.g., realized at the oral scanner and/or by or at a separate device, the display can be arranged to visualize feedback about the oral health (sensor) data relating to at least one oral health condition for at least two discrete positions or locations (or: segments), e.g., the display may be structured and/or arranged to visualize discrete position-resolved or discrete location-resolved (or: segment-resolved) oral health (sensor) data. The word sensor being in backets in “oral health (sensor) data” shall encompass oral health sensor data and oral health data. The display may be structured and/or arranged to show a depiction or visualization of at least a portion of an oral cavity, e.g., an abstract depiction or generalized depiction of at least a part of an oral cavity such as the dentition and the display may be arranged to additionally depict at least one feedback relating to the oral health (sensor) data and/or relating to the at least one oral health condition and/or to at least one condition class into which the oral health (sensor) data may have been classified with respect to the at least one oral health condition, which feedback may be realized by a change of the depiction or visualization of the at least portion of the oral cavity or by overlaying a visual representation of the discretely position-resolved or location-resolved (or: segment-wise) oral health data onto the depiction of the at least portion of the oral cavity or by depicting oral health data on the display, e.g., as text data, and relating it to a discrete position or location (i.e., segment) within the depiction of at least the portion of the oral cavity. The feedback and the depiction or visualization mentioned here typically occurs in a discretely position-resolved, i.e., segment-wise manner. While the present disclosure is focusing on either an abstract or on a more realistic depiction of at least a portion of the oral cavity such as the complete dentition, e.g., maxilla and mandibular, together with overlayed oral health data relating to one or more oral health conditions, this shall not exclude that the oral health (sensor) data is displayed in a different manner, e.g., as a table of oral health (sensor) data relating to one or several oral health conditions per discrete position or location within the at least portion of the oral cavity. It is noted that, e.g., an overlay of a live image or of a image computed from acquired images on a model of the dentition or the like is not to be considered a discretely segment-resolved feedback as such overlay leaves the analysis in segment information to the mind of a professional user. In the context of the present disclosure, feedback is provided in a processed manner such that a single indication or a single value per segment can be provided to a non-professional user without the need of any analysis to be made by the mind of the layman user.

The feedback relating to the oral health (sensor) data may occur “live” or in real time, e.g., while the user is using the oral scanner to perform a scanning procedure, which means that the feedback may be adaptive to the live progress of the scanning procedure, where “live” shall mean that there is only a short time delay between the acquisition step and the feedback step, e.g., a time delay of below 10 seconds or below 5 seconds or below 4 seconds or below 3 seconds or below 2 seconds or below 1 second. Feedback relating to the oral health (sensor) data may alternatively or additionally occur at the end of a scanning procedure by way of a summary feedback where the accumulated oral health (sensor) data is shown as a final result. Again, all feedback described herein shall be understood as including feedback that is discretely position-resolved or location-resolved (or: segment-wise). This may include a classification, preferably a discretely position-resolved or location-resolved (or: segment-wise) classification of the oral health (sensor) data with respect to at least two condition classes relating to the at least one oral health condition. Alternatively or additionally, the oral health (sensor) data and or the condition class determined in the classification step of the current scanning procedure may be compared with historic oral health (sensor) data and/or condition classes from a previous scanning procedure or from a sequence of previous scanning procedures and a trend or development of the oral health (sensor) data and/or the condition class over time may be visualized as feedback. Again, this may happen in a discretely position-resolved or location-resolved (or: segment-wise) manner. Such historic data may be stored in a memory that is coupled or connected with the processor. Stored historic data may include oral care activity data relating to at least one oral care activity procedure performed with an oral care device as will be discussed in more detail below.

The processor may be arranged to classify the oral health (sensor) data into at least two different condition classes relating to the at least one oral health condition, e.g., two condition classes relating to the severity of the oral health condition. The processor may be arranged to preferably classify the oral health (sensor) data in a discretely position-resolved or location-resolved manner (or: segment-wise), i.e., where the classification is done for a first position or first location or first segment such as the upper right molars and also for at least a second position or second location or second segment such as the lower left molars or the front teeth. Potential subdivisions of the oral cavity into discrete positions or locations or segments are in more detail discussed further below. By means of examples, the oral cavity that is intended for being scanned may be the dentition. Possible segments/discrete positions or locations may be (a1) upper yaw and lower yaw or (a2) mandible and maxilla or (b) upper right molars, upper front teeth, upper left molars, lower left molars, lower front teeth, and lower right molars or (c) buccal surfaces of the right upper molars, biting surfaces of the right upper molars, and lingual surfaces of the upper right molars or (d) buccal, lingual and chewing surfaces of tooth no. 26 of the human dentition or € one of the above and the tongue surface. All surfaces of all teeth of the human dentition may then lead to 72 segments (molars have two surfaces, while canine teeth and incisors have two surfaces) or to 84 segments in case of all wisdom teeth being included as well. In some examples, a complete scanning of the users dentition is intended as a standard scanning procedure while is some examples a scanning procedure only affects a selection of these segments completely covering the human dentitions. The latter may be specifically the case if after a previous scanning sessions and/or after a previous oral care activity only a selection of the segments covering the complete dentition is chosen for repeated scanning or focus scanning.

The terms ‘discrete position’ and ‘discrete location’ or ‘segment’ are used herein as being interchangeable. For sake of readability, the disclosure may not always refer to all three phrases in all instances.

The processor may be structured and/or arranged to process the sensor data in a “live” manner, e.g., during the scanning procedure so that “live” or, commonly speaking, real-time information about the progress or status of the scanning procedure and/or the progress or status of the oral health data acquisition can be visualized as feedback on a display as was already mentioned. The live display may as well comprise an abstract or more realistic depiction of at least a portion of the oral cavity and of overlayed feedback relating to at least the status of the scanning procedure. E.g., the various discrete positions or discrete locations or segments of the oral cavity to be scanned may be individually highlighted in a graded or staged manner so that the user can easily identify where the oral scanner still needs to be moved to or positioned to complete the scanning procedure. As an example, the depicted at least part of the oral cavity may be shown in a start color, e.g., dark blue, and the individual portions relating to different discrete positions or locations of the depicted at least part of the oral cavity may gradually be depicted in a brighter color until they are essentially white to indicate to the user a partially complete or finally complete scanning procedure with respect to the indicated discrete position or location of the oral cavity. The feedback relating to scanning procedure progress may be solely derived from position sensor data, e.g., from the accumulated time the oral scanner has performed a scanning procedure at the individual discrete positions or locations. This shall not exclude that the processor is structured and/or arranged to determine the scanning procedure progress in a more elaborated manner, e.g., by checking whether images taken by a camera preferably being part of the oral health sensor from the respective discrete position or location of the oral cavity comprise a sufficiently complete coverage of the discrete position or location of the oral cavity and/or whether such images have a certain image quality, e.g., are not blurred or unfocused or the like. The feedback relating to the scanning procedure progress may further comprise the overlay of position-resolved or location-resolved oral health (sensor) data onto the abstract or more realistic depiction of the at least portion of the oral cavity. It shall be understood that the overlaying of visualized feedback for displaying it on a display means the generation of a single image that is displayed on the display by a display controller. Overlaying means here that a base image, e.g., a depiction of a dentition, is amended to reflect the additional feedback that shall be provided.

The various components of the oral scanner system, e.g., the oral scanner, the processor, a separate display, a charger, and/or an oral care device may be arranged for data exchange or, more generally, for communication between at least two of these components in at least a unidirectional manner, preferably in a bidirectional manner. While such a data exchange or communication may be realized by a wired connection, e.g., when the processor is housed inside of the oral scanner, it is preferably realized by a wireless communication if the data exchange should occur between separate components. Then one of the components of the oral scanner system, e.g., the oral scanner, comprises a scanner communicator such as a transmitter or a transceiver and the other component, e.g., the processor realized in or by a separate device, comprises a processor communicator such as a receiver or a transceiver that may employ a proprietary or a standardized wireless communication protocol such as a Bluetooth protocol, a Wi-Fi IEEE 802.11 protocol, a Zigbee protocol etc. Each of the components of the oral scanner system may be arranged for communication with one or several other components of the oral scanner system and/or may be arranged for wireless communication with an Internet router or another device such as a mobile phone or tablet or a computer to establish a connection with the Internet, e.g., to send data to a cloud server that may be part of the oral scanner system and/or to receive data from a cloud server or any Internet service such as a weather channel or a news channel. That means that the oral scanner system may be arranged to communicate with the Internet directly or indirectly by a detour via a device not being a part of the oral scanner system.

It is also contemplated that the (position-resolved or location-resolved) oral health (sensor) data and/or the (position-resolved or location-resolved) condition classes are communicated from the oral scanner and/or the processor to an oral care device such as an electric toothbrush, a gum massager, an oral irrigator, a flossing device, a scaler, a tooth polishing device, a tooth whitening device, or the like. It may also be contemplated that the processor may communicate control data to the oral care device so that the oral care device is enabled to select one of at least two operational settings based on the control data, preferably in a discretely position-resolved or location-resolved manner. The latter requires that also a discrete position or location at which the oral care device is currently performing an oral care activity procedure is determined or tracked or monitored. An oral care device position sensor may be used for this task and reference is made to the description of discrete position or location determination of the oral scanner as the principles are the same.

### Oral Scanner Hardware—Attachment

Various hardware components of the oral scanner have already been described. In addition, the oral scanner may comprise an attachment that preferably is arranged to be replaceable so that different attachments can be used for different users or for different applications. One focus of the present disclosure is an oral scanner comprising an oral health sensor that comprises a camera as sensor receiver and at least a first light source as sensor emitter (see also description further below). A light inlet for the camera and a light outlet of the at least first light source may be provided at a head of the oral scanner. The attachment may then be realized as a detachable distance attachment. The distance attachment may be arranged to enable a scanning procedure with an essentially constant distance between the object or the objects that are being scanned, e.g., teeth, and the light inlet of the camera. A distance piece of the distance attachment may stay in contact with the object being scanned—specifically an outer surface of the object—to maintain the constant distance. The camera may have a focal length that creates sharp images of objects that have the distance to the light inlet of the camera that is defined by the distance piece. The distance piece may be realized as a closed wall element that surrounds the light outlet of the first light source and the light inlet of the camera so that the closed wall element effectively blocks ambient light from illuminating the currently scanned object and thus from eventually reaching the camera. The distance attachment can thus solve two objects, namely, to maintain a constant distance during the scanning procedure and to effectively block ambient light from reaching the object surfaces to be scanned. The latter being of particular benefit for embodiments where the light emitted by the first light source shall mainly be responsible for the oral health sensor data, i.e., image data outputted by the camera.

The attachment, e.g., the distance attachment, may be detachable to allow replacing the attachment when it is worn out or to allow changing the attachment if different attachments are used by different users of the oral scanner. The attachment may also be detachable to improve accessibility of parts of the oral scanner that benefit from regular cleaning such as a window covering the light outlet of the first light source and/or the light inlet of the camera. Also, the detachable attachment itself may benefit from regular cleaning, which cleaning becomes simpler when the attachment is detachable. E.g., the attachment may be immersed into a cleaning liquid to clean and to potentially sterilize it.

### Oral Health Sensor

The oral scanner as proposed herein comprises at least one oral health sensor and may comprise two or more different oral health sensors. The oral health sensor is understood to be a sensor that is arranged to acquire and output oral health sensor data relating to at least one property of the oral cavity that is relevant for determining a status of an oral health condition or that may be a direct measure of an oral health condition. The oral health condition may relate to the presence of at least one of the following: plaque, calculus (tartar), decalcification, white spot lesions, gum inflammation, tooth discoloration, stains, gingivitis, enamel erosion and/or abrasion, cracks, fluorosis, caries lesions, molar incisor hypo-mineralization (MIH), malodor, presence of germs such as pathogenic germs or fungi causing candidiasis, tooth misalignment, periodontal disease or periodontitis, peri-implantitis, cysts, abscesses, aphthae, and any other indicator that a skilled person would understand to relate to an oral health condition.

It shall be understood that the oral scanner may be arranged to acquire the oral health sensor data in a position-resolved or location-resolved manner where this is possible, e.g., malodor may be an oral health condition that affects the whole oral cavity, and which may thus not sensibly be acquired in a position-resolved or location-resolved manner. The latter shall not exclude that malodor is nonetheless acquired in a position-resolved or location-resolved manner and that also feedback relating to this oral health sensor data may be provided in a position-resolved or location-resolved manner, e.g., where then the feedback for all discrete positions or locations has the same malodor level or respective condition class.

Several of the above-mentioned oral health conditions can be detected by visual analysis, which typically requires an optical oral health sensor such as a camera and a software implemented on the processor arranged for determining the oral health condition and potentially also for assessing the level of severeness of the oral health condition based on the oral health sensor data provided from the optical oral health sensor, e.g., based on a classification of image data or a sequence of images with respect to at least two condition classes. Without limitation by theory, a classification of an input image may be done by a neural network such as a convolutional neural network (CNN), which preferably was trained with training images and relating condition class results. A classifier used by the processor may be directly fed with the oral health sensor data, e.g., image data, or the oral health sensor data may first be processed by the processor to determine, e.g., one or several features that herein are also called oral health data relating to at least one oral health condition.

The oral health sensor may include only a sensor receiver that acquires oral health sensor data by using an external medium such as ambient light or saliva or gaseous components present in the oral cavity etc. In accordance with several aspects, the oral health sensor may include at least one sensor emitter providing a primary medium and at least one sensor receiver that is arranged to detect at least the primary medium and/or a secondary medium created by interaction of the primary medium with the oral cavity, e.g., by interaction with oral cavity tissue. This shall not exclude that the sensor receiver is simultaneously also sensitive to an external medium as previously discussed. In a more specific example that is described in more detail below, the at least one sensor emitter is a narrow band light source emitting light of a certain wavelength range as primary medium and by interaction of this emitted light with certain material present in the oral cavity a second medium, namely fluorescence light of a higher wavelength may be created. The oral health sensor may then further include at least one sensor filter that filters out at least a portion of the primary medium and/or at least a portion of the secondary medium prior to the respective medium reaching the sensor receiver. It seems obvious that the sensor receiver may then as well be sensitive to ambient light that can pass the at least one sensor filter. The influence of the ambient light on the data acquisition can be reduced by specific measures such as a distance attachment discussed above. In some embodiments, the oral health sensor is an optical sensor such as a photodiode, an M times N array of light sensitive elements or a camera.

In accordance with some aspects, the oral scanner comprises an oral health sensor having at least a first light source and at least one camera, the oral scanner being structured and/or arranged for performing a scanning procedure, which typically is an optical scanning procedure, where optical scanning procedure here refers to a procedure in which a sequence of images is captured by the camera. The first light source may comprise a light outlet and the camera may comprise a light inlet, the light outlet and the light inlet may be provided at a head of the oral scanner. This may allow to arrange, e.g., a light-sensitive sensor element array such as an M times N light-sensitive sensor element array of the camera at a distance to the light inlet such as in the handle and to guide the light from the light inlet to the light-sensitive sensor element array by means of optical elements such as one or more lenses, one or more mirrors and/or one or more prisms and/or one or more lightguides etc. A user-operable input element may be provided at the oral scanner that upon operation by the user may initiate the optical scanning procedure. The oral scanner may comprise two or more cameras that may be arranged to allow a three-dimensional scanning of the at least portion of the oral cavity.

The oral scanner may comprise a second light source and potentially further light sources. The different light sources may use the same light outlet, or each light source may have its own light outlet. The first light source may emit light of a first wavelength or having a first wavelength range and the second light source may emit light of a second wavelength different to the first wavelength or light having a second wavelength range that does not or only partly overlap with the first wavelength or first wavelength range of the first light source. Additionally or alternatively, the first and the second light sources may be arranged to emit different light intensities. But this shall not exclude that a first and a second light source are provided to emit light of essentially the same wavelength or having the same wavelength range and also of essentially the same intensity. As one example, the first light source may emit light having a wavelength of or comprising a dominant wavelength of about 405 nm and the second light source may emit “white” light, i.e., light essentially covering the complete visual wavelength range between 400 nm and 700 nm or comprising several dominant wavelengths so that a human may consider the color impression of the emitted light as essentially white. The light sources are not limited to light sources that emit light in the visual range and any light source that emits in the infrared (IR) or ultraviolet (UV) wavelength range or that at least comprises wavelength ranges that extend into these areas is considered as well. The first and/or second light source (and any further light source) may be realized by a light emitting diode (LED), but also other light sources are contemplated, e.g., laser diodes, conventional light bulbs-specifically incandescent light bulbs, halogen light sources, gas discharge lamps, arc lamps etc.

The camera may comprise an array of light-sensitive sensor elements, where each light-sensitive sensor element may be arranged to output a signal indicative of a light intensity impinging onto a light-sensitive area of the light-sensitive sensor element. While each of the light-sensitive sensor elements may have an individual sensitivity range, i.e., individual wavelength sensitivity, the light-sensitive sensor element array may typically comprise light-sensitive sensor elements that all have about the same light sensitivity (ignoring differences in gain and the like as are typical and which are dealt with by calibration). The array of light-sensitive sensor elements may be realized as a regular M times N array even though this shall not exclude that the light-sensitive sensor elements are arranged in a different manner, e.g., in coaxial circles or the like. The array of light-sensitive sensor elements may be realized as a CCD chip or a CMOS chip as are typically used in digital cameras. The number of light-sensitive sensor elements may be chosen in accordance with the needs and the processing power of the processor. A resolution of 640 time 480 may be one choice but essentially all other resolutions are conceivable, e.g., the camera may be a 4K camera having a 3840 times 2160 resolution or the camera may have a lower resolution, e.g., a 320 times 240 resolution. It shall not be excluded that the camera comprises a line sensor as is typically used in a paper scanner.

In the context of the present application, a light-sensitive sensor element encompasses RGB sensor elements, i.e., each RGB-type light-sensitive sensor element would then deliver three signals that relate to the R (red), G (green) and B (blue) color channels.

The camera of the oral scanner may comprise further optical elements such as at least one sensor lens to focus the light onto the array of light-sensitive sensor elements, even though this shall not exclude that the camera is realized as a pinhole camera. The camera may also comprise at least one sensor mirror that guides the light onto the array. Further, the camera may comprise at least one sensor filter to selectively absorb or transmit light of a certain wavelength or light in at least one wavelength range. The at least one sensor filter may be fixed or may be moveable, i.e., the sensor filter may be arranged for being moved into and out of the light path of the camera. Several sensor filters may be provided to allow selective filtering of the light that should reach the array of light-sensitive sensor elements. The sensor filter may be a long-pass filter, a short-pass filter, a band-pass filter, or a monochromatic filter. The sensor filter may apply a wavelength-dependent filter characteristic so that a certain wavelength or wavelength range can pass but only at a reduced amplitude, while another wavelength or wavelength range may pass without attenuation and an even other wavelength or wavelength range may be completely blocked. The sensor filter may be realized as a colored filter or as a dichroic filter.

The first light source may be a narrow-band light source such as an LED. The narrow-band light source may emit light in the range of between 390 nm and 410 nm (FWHM) such that a wavelength of about 405 nm is at least close to the dominant wavelength of the LED. Light of around 405 nm causes fluorescence light to be emitted by tooth enamel and by plaque as was already mentioned. A sensor filter may then be used that transmits only light having a wavelength above about 430 nm, preferably the sensor filter may be a cut-off filter having a cut-off wavelength of 450 nm allowing light of greater wavelength to pass towards the light-sensitive sensor element array so that reflected light originating from the first light source is absorbed and only fluorescence light transmitted by the sensor filter is determined.

The camera may be realized by a camera module as is available, e.g., from Bison Electronics Inc., Taiwan. Without any limitation, the camera module may comprise a light-sensitive sensor array having an M times N pixel count of 1976 times 1200 (i.e., a 2.4 megapixel chip) realized in CMOS technology, but not all pixel may necessarily be used for capturing images during a scan. The camera module may comprise a lens causing a 12.5 mm focal length so that sharp images can be captured of objects close to the camera. This shall not exclude that an autofocus camera is used. A hyperspectral imaging camera may be used as well.

The examples relating to optical sensors, specifically cameras, are not to be understood as limiting. The at least one oral health sensor may also be realized as one from the group comprising, in a non-limiting manner, temperature sensors, pressure sensors, pH sensors, refractive index sensors, resistance sensors, impedance sensors, conductivity sensors, bio sensors such as sensors comprising a biological detection element, e.g., an immobilized biologically active system, that is coupled with a physical sensor (transducer) that converts the biological-chemical signal into an electrical or optical signal and typically includes an amplifier etc.

As was already explained, the oral health sensor acquires and outputs oral health sensor data that are transmitted in the form of analog or digital signals and the processor may be arranged to process the oral health sensor data to determine oral health data and/or condition class data, preferably condition class data relating to the oral health condition.

### Position Sensor

The term “position sensor” shall encompass all position sensor arrangements that can determine a discrete position or location or segment where the oral scanner head performs a scanning procedure at a given time instant in the oral cavity and may include such determination also with respect to at least one discrete position or location or segment that relates to the outside of the oral cavity. It shall be understood that the use of the term “position sensor” does not mean that the position sensor is itself able to directly determine a position inside or outside of the oral cavity, but that a discrete position or discrete location or segment inside or outside of the oral cavity can be derived from the position sensor data, e.g., by a deterministic computation based on the input from the position sensor, by a decision tree, by a clustering or by a classification algorithm, to name just a few. The processor may be structured and/or arranged to perform such discrete position or location or segment determination based on at least the position sensor data. An oral health sensor such as a camera may also provide position sensor data, i.e., the oral health sensor may additionally be used as position sensor or a further camera may be provided as position sensor. As one example, the image data provided by a camera provided at a head of the oral scanner may allow to determine the type of tooth that was imaged and to thus derive the discrete position or discrete location or segment in the mouth that was scanned (see reference to EP 2 189 198 B1 below).

Document EP 3 141 151 A1 describes, inter alia, a location determination based on a fusion of image data from a camera acquiring images of the user while performing an oral care activity with an oral care device, which camera is separate from the oral care device, and of data from an accelerometer disposed in the oral care device to determine the orientation of the oral care device relative to Earth's gravitational field. Based on the one hand on a classification of the image data created at a given time instant by machine learning algorithms that are each specifically trained for one of the locations and on the other hand on the classification of the orientation angles determined from the accelerometer data at that same time instant, a fused location determination result is computed. The classification algorithms output values similar to probabilities for the plurality of locations within the oral cavity at which the oral care activity might be performed. The highest measure typically indicates with a certain reliability the location at which the activity is performed. EP 3 141 151 A1 shall be incorporated herein by reference. The position sensor in this example comprises a separate camera as a first position sensor and an accelerometer disposed in the oral care device (which might be the oral scanner in accordance with the present disclosure) as second position sensor. This indicates that the term “position sensor” does not refer to a single sensor arrangement but that “position sensor” encompasses embodiments using two or more different position sensors to provide position sensor data.

Document EP 3 528 172 A2 describes, inter alia, the determination of a discrete position or discrete location or segment within the oral cavity at which an oral care activity is currently performed, which determination relies on the classification of position sensor data that is a temporal sequence of inertial sensor data created by, e.g., an accelerometer and/or a gyroscope located in an oral care device by means of a neural network, preferably a recurrent neural network. Based on the trained neural network, the classification of a current temporal sequence of position sensor data provides a set of values similar to probabilities with respect to the plurality of possible discrete positions or locations within the oral cavity. The highest value typically indicates the location at which the activity is performed. EP 3 528 172 A2 shall be incorporated herein by reference.

Each of the above-mentioned technologies and the technologies mentioned in the following paragraphs of the present section can be used for the determination of the discrete position or location within the oral cavity at which the oral scanner in accordance with the present disclosure is performing a scanning procedure, but other technologies may be used as well. E.g., it is known to track the positions of a user's head and of a toothbrush in a calibrated magnetic field or to use ultrasound emitters at a user's head and at a toothbrush to track the motion of both of them in a calibrated ultrasound receiver arrangement so that the relative position of the toothbrush with respect to the user's head and hence with respect to the user's oral cavity can be determined. Likewise, IR emitters and receivers may be used. Further technologies may be used as well, e.g., motion tracking technologies using a plurality of cameras known from CGI movies.

The latter mentioned technologies can determine a discrete position or location at which the oral care activity, e.g., toothbrushing, is performed with a relatively high precision (e.g., on the level of individual teeth), which precision may justify the use of the term ‘position’ (while this position is still mapped onto a ‘segment’, where the segment may still represent a single tooth or a group of teeth). The technologies described in the paragraphs before have, at least at the time of filing the present disclosure, not been developed to deliver results at such a high precision and may allow to determine one of 16 different segments in the dentition at which the oral care activity is performed. Then the term “location” may be better suited as the determination typically relates to a group of teeth (e.g., the left upper molars) or to a group of surfaces of a group of teeth (e.g., the buccal surfaces of the right lower molars). In a more common sense, the term “segment” is used to indicate a discrete position or a discrete location.

Document EP 2 189 198 B1 describes the determination of a discrete position or location in the oral cavity by analyzing camera data from a camera located at a toothbrush head. It is described that the analysis of image data can identify the tooth that is shown on the image. One may contemplate to train a classifier with labelled images of the teeth and/or other portions of the oral cavity of the user so that the processor can reliably identify the position in the oral cavity at which the scanning procedure is currently performed.

Document US 2010/0170052 A1 describes the determination of a discrete position or location in the oral cavity at which an oral care activity is performed by an oral care device by analyzing images from a separately positioned camera that images the user's face and the oral care device. EP 2 189 198 B1 and US 2010/0170052 A1 shall be incorporated herein by reference.

### Processor Hardware

The processor may be any kind of general-purpose integrated circuit (e.g., IC; CPU) or application specific integrated circuit (e.g., ASIC) that may be realized by a microprocessor, a microcontroller, a system on chip (SOC), or an embedded system etc. The processor shall not be understood as necessarily being a single circuit or chip but it is contemplated to provide the processor in a distributed manner where one part of a processing task may be performed by a first processor sub-unit and one or several further processing task(s) may be performed by at least a second or several further processor sub-unit(s), where the different processor sub-units may be physically disposed at different locations, e.g., in or at the oral scanner, in or at a remote device and/or in or at a cloud computer etc. The processor may be essentially completely realized by a cloud computing device. It shall also be included that the processor may comprise analog circuit elements and integrated circuit elements or only analog circuit elements.

The processor has at least one input and at least one output. The processor receives sensor data and/or oral care activity data from an oral care device via the input and outputs oral health data and/or condition class data and/or control data, preferably discrete position or location resolved oral health data and/or condition class data and/or control data via the output. Condition class data refers to data that classifies oral health (sensor) data into at least one of at least two condition classes, e.g., into a not severe class and a severe class or into more than two classes, e.g., into a not severe class, into a to be monitored class and in into an oral care professional visit recommended class. The latter examples are for exemplification and a skilled person may use any other number of classes and may name these classes appropriately.

### Processor Software: Classification

It has been described in various previous paragraphs that the processor is structured and/or arranged to classify the oral health sensor data and/or oral health data into at least two condition classes. In a more mathematical language, the oral health (sensor) data (preferably for a given discrete position or location) may be said to be an observation and the condition classes to be categories and a classifier algorithm may then be used to decide to which of the categories the observation belongs to. The oral health (sensor) data may comprise one or several variables or features characterizing the oral health condition, e.g., the oral health data may comprise a normalized area of plaque per considered discrete position or location. The classifier may then simply label the input feature (size of plaque) into the categories by comparison with one or several threshold values. The threshold value(s) themselves may be derived from expert opinions or from an analysis of oral health conditions of a plurality of subjects by means of a machine learning algorithm. Instead of using a feature or a vector of features derived from the oral health sensor data, the oral health sensor data may be used as input into a classifier without any prior processing, e.g., a neural network may be directly fed with the image data acquired by an oral health sensor comprising a camera.

Threshold values or other parameters affecting the classification may be set to different values for different discrete positions or locations in the oral cavity. Such a discrete position or location dependent threshold value or parameter affecting the classification for a given oral health condition may preferably be influenced by at least one from the non-limiting list including the discrete position or location in the oral cavity in a global sense (i.e., for all users) or for an individuum, a history of evolvement of the oral health (sensor) data or the condition class relating to this discrete position or location for the given oral health condition, or an overall or average oral health condition status for a given user.

While the above-described threshold-based approach for the classification may be sensible for oral health data comprising one or two features per oral health condition, a different classifier algorithm may be used in case the oral health data comprises a plurality of features. E.g., a neural network may then be applied for the classification task, or any other classification algorithm known to a skilled person. The classification algorithm may be chosen to be one from a non-limiting list comprising: linear classifiers, support vector machines, quadratic classifiers, kernel estimation, boosting, decision trees, neural networks, transformers, genetic programming and learning vector quantization.

Condition classes may be determined for at least one of the at least two discrete positions or locations, preferably for all the discrete positions and locations that are used to subdivide the at least portion of the oral cavity that is scanned into segments. At each such discrete position or location at least two condition classes may be defined, preferably at least three condition classes may be used (similarly to a traffic light system that either shows a green, a yellow, or a red light). The underlying threshold values or parameters used by a classifier algorithm may be adaptive and may thus change over time and may be different for different users.

### Processor Software: Temporal Assessment

In accordance with some aspects, the oral scanner system proposed herein is intended for a regular repetition of a scanning procedure such as an optical scanning procedure of at least a portion of the oral cavity of a user or of a treated subject to thereby create new oral health (sensor) data. The oral scanner system may preferably be structured and/or arranged to compare newly determined oral health (sensor) data and/or condition class data with previously created oral health (sensor) data and/or condition class data and to update information about the temporal development of the oral health (sensor) data and the condition class data, which may then lead to updated information to be fed back to the user. The comparison process may result in comparison data and/or in discretely position-resolved or location-resolved comparison data. The processor may comprise a memory for storing and later accessing previously and currently acquired oral health sensor data and/or position sensor data and any data created by processing such data, e.g., oral health data and/or condition class data and/or discretely position-resolved or location-resolved oral health sensor data and/or discretely position-resolved or location-resolved condition class data and further may also comprise comparison data and/or discretely position-resolved or location-resolved comparison data. Stored data relating to a previous scanning procedure is also referred to as historic data. In addition to the just mentioned data, further data may be stored in the memory, e.g., historic scanning procedure progress data or historic oral care activity data relating to a previous oral care activity procedure performed by an oral care device, which oral care activity data may have been transmitted to the processor and be stored in the memory. The historic data stored in the memory may also be used by the processor to adapt a next scanning procedure, e.g., to adapt at least one scanning procedure parameter and/or to adapt a scanning procedure guidance, which comprises at least one automatic feedback to be provided to the user immediately prior to or during a next scanning procedure. That means that the scanning procedure guidance is not indicted at the end of the current scanning procedure but is automatically indicated just immediately prior to the next scanning procedure so that the user can basically benefit from such a guidance in the scanning procedure that is about to be initiated. The scanning procedure guidance may have been determined in a segment-resolved manner, i.e., for each of the discrete positions or locations in the oral cavity. Then, such a segment-resolved scanning procedure guidance may be automatically indicated once the user reaches the respective segment.

### Display

The oral scanner system may comprise a display as a feedback element of a feedback unit, preferably a display allowing visual depiction of oral health data and/or condition class data and/or oral scanning progress data. The display may be any type of display such as an LCD, LED, OLED (PMOLED or AMOLED) etc. display. The display may be a monochromatic or color display. The display may have any suitable resolution such as a 96 times 48 resolution for a display implemented on the oral scanner or may comprise custom-made illuminable areas. As a display of a user device such as a mobile phone, table computer, laptop, smart watch etc. may be used, the respective technologies and resolutions of the displays of such user devices are to be considered. In such a case an App or software running on such a device may provide the relevant programming for the general-purpose processor of the user device to function at least as one processor sub-unit or as the processor in accordance with the present disclosure. The respective App or software may also implement any display control needed to visualize the information as discussed herein.

The present discussion of a display shall not exclude that the oral health (sensor) data and the scanning procedure progress data etc. are additionally or alternatively fed back to the user by means of other feedback elements of a feedback unit such as a plurality of individual visual feedback elements and/or an audio feedback element and/or a haptic feedback unit as was already described. As an example, assuming that the oral cavity would be segmented into four positions and/or locations for which the scanning procedure is to be monitored and, alternatively or additionally, for which oral health data are to be fed back, then the scanning procedure progress data can be fed back by using four visual feedback elements that start at a first color, e.g., dark green, and that are controlled to gradually show a brighter green until the scanning procedure is considered as complete for a given discrete position or location and the light indicator might then, e.g., show a white signal. An RGB LED per light feedback element could be used for this. Similarly, the live communication of the oral health data, e.g., relating to plaque, could use four visual feedback elements as well and start at white to indicate no plaque and be gradually changed on a scale towards red to communicate the amount of plaque detected at the respective discrete position or location. Instead of a live feedback, the oral health data may be fed back to the user only at the end of the scanning procedure to indicate the levels of plaque identified in the scanning procedure. A classification of the oral health data relating to plaque may then be indicated with a flashing light for a condition class ‘severe’. A skilled person will understand how to modify the number of visual feedback elements, colors used and other means of feedback such as flashing, intensity variations etc.

### Display Software

It is envisioned that the display comprises a display controller that converts oral health (sensor) data, preferably position-resolved or location-resolved oral health (sensor) data and/or condition class data and/or scanning procedure progress data into a visualization that is shown on the display, where the visualization is called the feedback screen. The feedback screen may comprise at least one element of a graphical user interface. In the present disclosure, focus is put on a feedback screen that comprises a visualization of at least a portion of the oral cavity, which visualization may be a two-dimensional visualization or a 3D type of visualization, where the latter means a visualization on the two-dimensional display that provides a three-dimensional impression. The visualization of the at least portion of the oral cavity may comprise a visualization of the dentition, i.e., a visualization of the teeth of the dentition, which may be an abstract visualization or a more realistic visualization. The visualization may be based on a generic model of a dentition or may take into account individual data from a user such as missing teeth or the like. An abstract visualization of the complete dentition may comprise a circle or an annulus, where the top of the circle or annulus visualized on the display may be understood to represent the upper front teeth and the bottom of the circle or annulus may represent the lower front teeth, while the sides then represent the left and right molars, respectively. Instead of a continuous circle or annulus, a plurality of segments of a circle or an annulus may be visualized, e.g., an upper about 180-degree segment and a lower about 180-degree segment may indicate the maxilla and the mandible, respectively. Or four about 90-degree segments may be used to display quadrants of the dentition, which is known to the skilled person from, e.g., the visualization on the Oral-B SmartGuide. Further, six segments may be used. Is may also be contemplated to visualize each tooth of a generic or individualized dentition by a single segment or to use any other kind of segmentation that would seem appropriate to a skilled person. At least one of the segments may be separated into at least two areas that may represent inner and outer tooth surfaces, preferably three areas that represent inner and outer tooth surfaces (such as buccal and lingual surfaces) and the biting or occlusal surface, which may be particularly sensible for molars and wisdom teeth. This shall not exclude any other type of fragmentation of the segmented visualization. While here the segments were described as portions of a circle or an annulus, it is also contemplated that segments may be visualized in a different manner. E.g., each tooth may be represented by a circle or a segment representing a plurality of teeth may be visualized as a plurality of overlapping circles, where the number of circles may coincide with the number of teeth that typically are represented by this segment, even though this is not to be understood as limiting. The visualization of the dentition may comprise information as is used in accordance with ISO 3950:2016. Some example visualizations will be discussed further below with reference to the figures.

Instead of an abstract visualization, a more realistic depiction of the dentition may be chosen, e.g., up to 32 teeth for the permanent dentition of a grown-up user or up to 24 teeth for the primary dentition of a child. As was already mentioned, the visualization may be individualized, e.g., a user may be able to input personal tooth characteristics such as missing teeth, misaligned teeth, fillings, inlays, crowns, artificial teeth, braces etc. that may be taken into account in the visualization. As will be explained further below, the user may also be allowed to provide information about the oral heath health condition of at least one surface of a tooth, at least one tooth, a group of teeth or the complete dentition and/or about the gums. E.g., a user may provide input about tooth discoloration or braces or cavities etc., where the oral scanner and/or the separate device may provide an interface for inputting information. Instead of a manual input, the oral scanner may be structured and/or arranged to perform a scanning procedure in which relevant information of the oral cavity is acquired to individualize a visualization at the at least portion of the oral cavity in an automated manner. While the mentioned interface may be realized as a graphical user interface, this shall not exclude that the user can additionally or alternatively provide input by a voice recognition interface and/or a keyboard etc. The interface may also allow the user to input personalization information, e.g., a name, an email address or the like and/or may allow dedicated access by a dentist to any stored data, where the latter may preferably be allowed by means of a remote access, e.g., from a computer at a dentist's office.

The above shall not exclude that the visualization of at least a portion of the oral cavity further comprises the tongue, preferably various areas of the tongue, the inner cheeks, the lips, the uvula, the pharynx, the palate etc. In some visualizations at least one of the previously mentioned portions and at least one portion of the dentition is visualized, such as the tongue and the complete dentition.

This abstract or more realistic visualization of the at least portion of the oral cavity provides a map onto which further data such as oral health data or scanning procedure progress data may be visualized in a manner that the user can relate the additional information to a location or position within the oral cavity.

The mentioned visualization may be used in manifold feedback applications. E.g., the visualization may be used to provide feedback about the scanning procedure progress in real-time, i.e., in a live manner, which means that the discrete position or location at which the oral scanner is currently performing a scanning procedure and the respective visualized segment or segments relating to this discrete position or location may then be amended so that the scanning procedure progress can be understood by the user. The visualized segment at which a scanning procedure is performed may be additionally visually highlighted, e.g., by a halo or similar visual measures to allow a user to immediately identify where the oral scanner performs scanning. An example where the coloring of the respective segments starts at a first color that is gradually changed to a second color was already discussed (white and black are here understood to be colors). While this example referred to a gradual change from one color to another color, it shall be understood that this is not limiting. E.g., start and end colors may be chosen to be different for different segments. It is not necessary to have a gradual change. A stepwise change or a single step from the start color to the end color is also envisioned. Further, instead of or additionally to colors, segments may comprise a start pattern and an end pattern to visualize the scanning progress.

Interaction with an Oral Care Device

As was already mentioned, the oral scanner system may comprise an oral care device such as an electric toothbrush, an electric flossing device, or an electric irrigation device etc. that is provided to perform an oral care activity such as teeth cleaning, interdental area cleaning, gum massaging etc. The oral care device may preferably be equipped with its own oral care device position sensor (e.g., an IMU sensor) so that its discrete position or location in the oral cavity where an oral care activity procedure such as tooth brushing or flossing, or irrigation is performed can be determined independently from the determination of the discrete position or location of the oral scanner. Additionally or alternatively, the discrete position or location where the oral care device performs an oral care activity procedure may at least in part be determined by using the same position detector that serves to determine the discrete position or location of the oral scanner (e.g., by the same external camera), i.e., the position sensor of the oral scanner may be a shared position sensor.

The use of a sole oral scanner for performing an oral scanning procedure on the one hand and a sole oral care device for performing an oral care activity is the interaction between the oral scanner and the oral care device. E.g., the oral scanner may provide control data to be received by the oral care device, which control data will affect the oral care activity insofar at least one oral care guidance is triggered by the control data or at least an operational parameter is influenced by the control data. The control data may in particular cause the guidance or influence to happen in a discretely position-resolved or discretely location-resolved or segment-wise manner. The sole oral scanner can be used to perform a dedicated scanning procedure that is not influenced by any parallel oral care activity and the oral care device can be used to perform a dedicated oral care activity that is not disturbed by any parallel scanning procedure. Data collected during the oral care activity may likewise be used to determine control data that can be send to the oral scanner to influence the next oral scanning procedure, e.g., can limit or focus the scanning to segments that were not properly cared for in the oral care activity. An oral care system comprising an oral scanner and an oral care device adds benefits to a simple juxtaposition of the two devices.

The oral care device may comprise a device communicator such as a receiver or a transceiver for at least receiving control data from the processor via the processor communicator, which control data may specifically be used to select one from at least two different operational settings of the oral care device, preferably wherein the control data is used to select one from at least two different operational settings in a discrete position or location dependent manner, i.e., in a segment-resolved manner. Such an operational setting may relate to a recommended time for performing an oral care activity procedure either in general or at a particular discrete position or location or may relate to a recommended minimum and/or maximum pressure or force value to be applied by an oral care head either in general or at a particular discrete position or location or may relate to feedback to be provided to the user either in general or when a particular discrete position or location is treated or may relate to an operational mode to be used in general or at a particular discrete position or location and where the oral care device may then be arranged to automatically switch into this mode due to the control data that was received. An operational mode may preferably be a motion mode at which an oral care head of the oral care device is driven and may include at least one parameter from a list including velocity, frequency, and amplitude.

### Example Embodiments

Without wanting to be limiting, the present disclosure focuses on an oral scanner system comprising an oral scanner having an oral health sensor and a processor and preferably a position sensor. The oral scanner is structured and/or arranged to perform a scanning procedure of at least a portion of the oral cavity, such as a portion of the dentition or of the complete dentition and/or of more or other portions of the oral cavity and to acquire oral health sensor data by means of the oral health sensor and preferably position sensor data relating to a discrete position or location (or: a segment) from a list of at least two discrete positions/locations or segments at which the oral scanner currently performs the scanning procedure. The processor comprises or is coupled with a memory of the oral scanner system in which historic scanning procedure data is stored from at least one previous scanning procedure, specifically historic oral health sensor data and/or historic oral health data and further preferably historic comparison data and/or historic classification data, where the latter two will be discussed more in detail below. The stored historic data is optionally stored in a discretely position/location-resolved manner (in a segment-resolved manner). The processor is preferably structured and/or arranged to assign the currently acquired oral health sensor data or the therefrom derived oral health data to the current discrete position/location or segment at which the oral scanner performs the scanning procedure to create current discretely position/location-resolved or segment-resolved oral health (sensor) data. While the memory may be realized in or at the oral scanner or in or at a separate device which may, e.g., realize a display as part of the feedback unit, the memory may also be disposed in a cloud and the memory may also be a distributed memory located at different physical locations. The processor is structured and/or arranged to compare the currently acquired oral health sensor data or the currently determined oral health data with the stored historic oral health sensor data or the stored historic oral health data, respectively, and to thereby generate comparison data, where comparison data may be a difference between the historic data and the current data, e.g., expressed as a percentage difference, or the comparison data may be a qualitative information that only indicates whether the comparison showed an increase or a decrease of the values being compared. These examples are to be understood as non-limiting. Comparison data may also include a determination of whether an oral health condition, e.g., expressed by means of a condition class, improved or worsened in view of the comparison with the historic data. Further, the oral scanner system comprises a feedback unit structured and/or arranged to provide user-perceptible feedback on the comparison data and may also provide user-perceptible feedback on the oral health sensor data and/or the oral health data. Comparison data is optionally additionally or alternatively, determined in a discretely position/location-resolved or segment-resolved manner.

The oral scanner system preferably comprises a position sensor that is structured and/or arranged to acquire and output position sensor data relating to the position or location in the oral cavity at which the oral scanner performs the scanning procedure at the present time instant or performed the scanning procedure at a given time instant, where here time instant includes a time period needed to acquire the oral health sensor data and the respective position data. In case of a time period, the center time may be used as time instant. The at least portion of the oral cavity may thus be divided into at least two positions or locations as was already discussed. The processor is structured and/or arranged to determine the discrete position/location or segment at which the oral scanner is or was performing the scanning procedure and to determine discretely position-resolved/location-resolved or segment-resolved oral health sensor data and/or discretely position-resolved/location-resolved or segment-resolved oral health data for each of the at least two discrete positions/locations or segments, where the respective oral health sensor data and/or oral health data is assigned to the determined discrete position/location or segment relating to the same instant. The comparison may then be done with respect to stored historic discretely position-resolved/location-resolved or segment-resolved oral health sensor data and/or stored historic discretely position-resolved/location-resolved or segment-resolved oral health data. The comparison result may then be fed back for at least one discrete position/location or segment, preferably for at least two discrete positions/locations or segments and further preferably for all discrete positions/locations or segments.

Examples of position sensors were already discussed. Inertial measurement units (IMUs) comprising an accelerometer and/or a gyroscope located at or within the oral scanner are contemplated, preferably those realized as a MEMS sensor. It is mentioned here again that an oral health sensor for acquiring oral health sensor data may simultaneously also serve as position sensor. The image data outputted by the camera may, e.g., be classified by a classifier algorithm to determine whether the image taken at a given time instant belongs to a certain position or location. As was discussed before, data from an IMU sensor may be in parallel classified and the results may be fused to determine the position or location or IMU data and image data or feature(s) derived from IMU data and/or image data may be inputted into a classifier algorithm. The oral health sensor may comprise an optical sensor such as an M times N array of light sensitive sensor elements and may be realized as a camera for taking images. While in some instances the oral health sensor data may already provide a direct insight into the oral health condition (e.g., reference is made to the discussion of a malodor sensor senor above), it is contemplated that the processor may be structured and/or arranged to process the oral health sensor data to determine oral health data that are a direct measure of the oral condition. E.g., in a case where the oral health sensor is a camera, the oral health sensor data is image data and the processor may need to process the image data to determine the oral health data, which may relate to plaque visible in the image or caries lesions or missing teeth or discoloration etc. Reference is made to the list of oral conditions previously discussed. The processor may further be arranged to classify the oral health sensor data or the oral health data with respect to at least two condition classes. The oral health data and the condition classification data relating to the classification results may in particular be determined for at least two of the at least two discrete positions/locations or segments. The processor may be structured and or arranged to compare the currently determined condition class or the discretely position-resolved/location-resolved or segment-resolved condition classes with at least one historic condition class or historic discretely position-resolved/location resolved or segment-resolved condition classes, respectively, that were determined in the course of at least one previous scanning procedure and which condition classes are stored as historic condition class data in the memory.

The feedback unit may be structured and/or arranged to provide feedback about the oral health data and/or the condition classification data, either during the scanning procedure and/or at the end of the scanning procedure. The feedback unit may comprise at least one feedback element for a visual, audible and/or haptic or tactile feedback relating to the oral health sensor data and/or the oral health data and/or the condition classification data, specifically in case such feedback is provided as discretely position-resolved/location-resolved or segment-resolved feedback. As was mentioned, the present focus is on the feedback provided about the comparison results, preferably in a discretely position-resolved/location-resolved or segment-resolved manner, to continuously guide the user to achieve an optimum usage of the oral scanner system. The intention is to provide an easy to digest simple feedback providing a single information such as a single number or value per segment to the user. The feedback unit may comprise at least two visual feedback elements for feedback of discretely position-resolved/location-resolved or segment-resolved feedback. The feedback unit may in particular comprise a display, where it shall be understood that a display may be used to define a plurality of visual feedback elements and reference is made to the respective discussion in a previous paragraph.

The feedback unit may be provided by a separate device such as a proprietary device (e.g., a charger with a display), a computer, a notebook, a laptop, a tablet, a smartphone, or a smart watch etc. The processor may at least partially be provided by the separate device. It is contemplated that separate units or devices as discussed herein may communicate in a wireless manner. E.g., each of the units or devices may comprise a communicator for establishing at least a unidirectional or a bi-direction or multi-directional wireless communication. As was already discussed, the feedback unit may provide an abstract or more realistic visualization of the at least portion of the oral cavity that shall be scanned, e.g., an abstract depiction of the dentition, which is taken as an example. The visualization of the dentition may be overlaid with a visualization of the scanning procedure progress data, the oral health (sensor) data and/or the condition classification data and/or the comparison data. The term “overlaid” should be understood to mean that a two-dimensional image may be displayed that is based on the depiction of the dentition and may comprise further information that is additionally depicted and/or of, e.g., colorations or patterns of at least portions of the depiction of the dentition. The image may comprise elements of a graphical user interface.

In accordance with one aspect, the present disclosure is concerned with an oral scanner system comprising an oral scanner having an oral health sensor comprising a camera for performing an optical scanning procedure and a processor for receiving image data from the camera and to compare the image data and/or oral health data relating to at least one oral health condition derived from the image data with historic image data and/or historic oral health data, which historic data is stored in a memory connected or coupled with the processor and to generate comparison data relating to a change in the image data and/or oral health data between the current optical scanning procedure and a previous optical scanning procedure, i.e., relating to the comparison result. The oral scanner system then further comprises a feedback unit for providing feedback about the comparison result. The oral scanner may comprise a position sensor as mentioned above so that discretely position-resolved/location-resolved or segment resolved comparison data can be created, and respective discretely position-resolved/location-resolved or segment-resolved feedback be provided.

Discussion of Embodiments with Reference to Figures

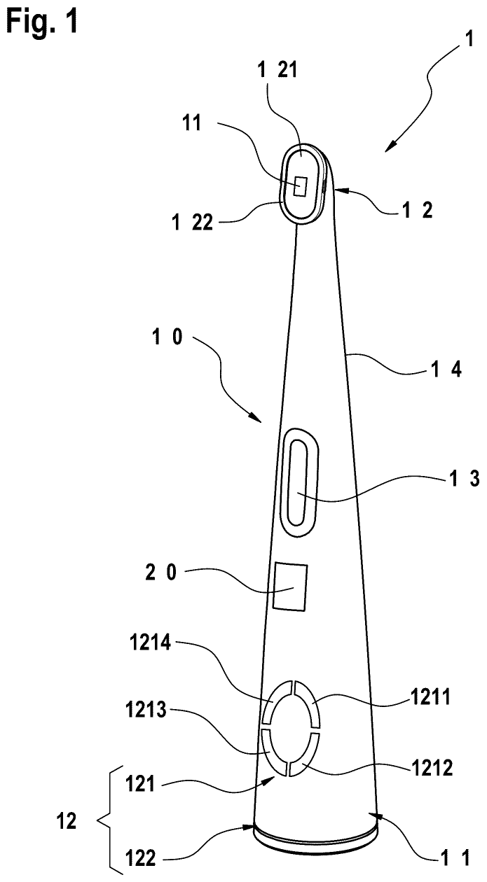

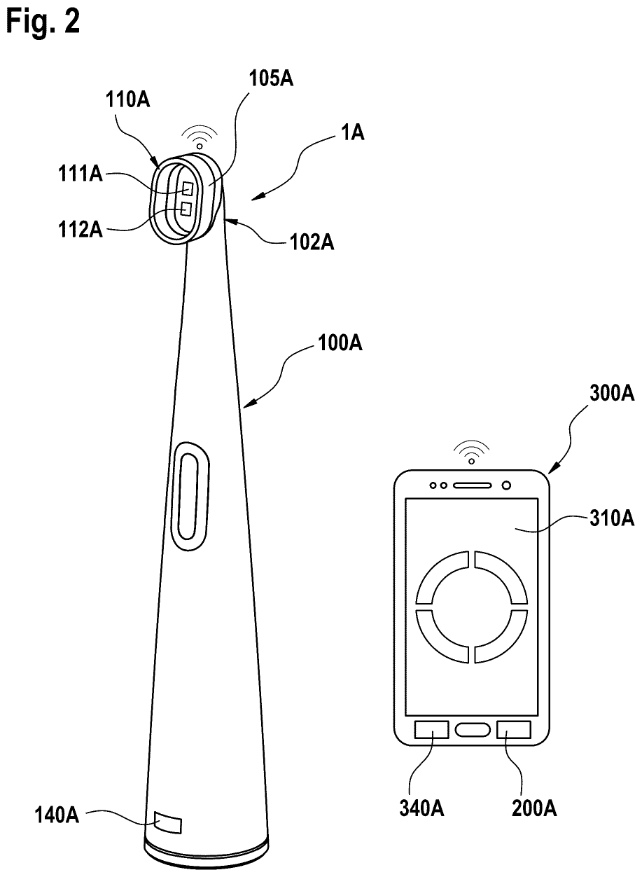

FIG. 1 is a schematic depiction of an example oral scanner system 1 in accordance with the present disclosure. The oral scanner system 1 comprises an example oral scanner 100 solely structured and arranged for performing oral scanning procedures without any oral care activity and a processor 200, where the processor 200 is in this example disposed at or inside of the oral scanner 100. The oral scanner 100 comprises a handle portion 101 and a head portion 102. An oral health sensor 110 is disposed in or at the oral scanner 100. Generally, two or more different oral health sensors may be used and may thus be disposed in or at the oral scanner 100. In the shown example, at least a measurement inlet such as a light inlet cooperating with the oral health sensor 110 is provided at the head portion 102 so that an acquisition of oral health data based on light measurements by the oral health sensor 110 is enabled at the head portion 102. The head portion 102 here comprises a flat transparent window 1021 surrounded by a frame structure 1022 that may be arranged and/or structured to receive a preferably detachable attachment (see FIG. 2). The head portion 102 is dimensioned such that it can be conveniently introduced into the oral cavity of a human or an animal. The handle portion 101 is dimensioned such that it can be conveniently grasped by a hand of a human user. The handle portion 101 and the head portion 102 may be separable from each other. In some embodiments, the handle portion 101 can be equipped with different replaceable head portions in addition to an oral scanner head portion such as a brush head portion or the like. The handle portion 101 may comprise at least one user operable input element 103 such as an on/off button and/or a selector button or switch. The oral scanner 100 has an outer housing 104 that may preferably be hollow to accommodate various internal components such as a preferably rechargeable energy source and a related charging circuit for preferably wireless charging of the energy source, a circuit board comprising various electronic components for the control of the oral scanner etc. Generally, the oral scanner 100 is structured and/or arranged for performing a scanning procedure of at least a portion of the oral cavity of a subject, i.e., while the user is holding and moving the oral scanner 100, the oral scanner acquires oral health sensor data and determines the progress of scanning and preferably analyses the acquired oral health sensor data with respect to the at least one oral health condition. While this is understood not to be limiting, the processor 200 may be disposed on said circuit board. The processor 200 is coupled or connected with the oral health sensor 110 for receiving signals from the oral health sensor 110, i.e., for receiving oral health sensor data at the processor 200. The processor 200 may be structured and/or arranged for processing the oral health sensor data to derive or determine oral health data that relates to at least one oral health condition, such as plaque. In some embodiments, the oral health sensor may output oral health sensor data that are a direct measure of the relating oral health condition so that only a limited processing of the oral health sensor data may be required (if at all), e.g., some reduction to an integer number or a computation of a normalized value etc. The processor 200 may also be structured and/or arranged to classify the oral health (sensor) data into at least two condition classes relating to the at least one oral health condition, e.g., into a ‘no oral health concern’ class (or ‘green’ class) and into an ‘oral health concern’ class (or ‘red’ class), which may be done based on a comparison with at least one threshold value. Reference is made to a respective previous paragraph in which the classification is explained in more detail. The classification may also be done with respect to at least three classes, e.g., besides the ‘green’ class a ‘low concern’ (‘orange’) class and a ‘high concern’ (‘red’) class may result from the classification process. It is again noted that one main aspect of the present application is the provision of simple feedback (e.g., a single value or a single color or a single pattern or the like) for each of the segments (discrete positions or locations) being scanned to the user by the feedback unit. This provision of simple feedback requires some processing of the oral health sensor data and/or of the position sensor data to determine a simple feedback (value) per segment. The simple feedback may be a number or a color or the like. In some embodiments the indicated color may vary in an essentially step-less manner to convey the feedback while in some embodiments the feedback may be limited to a binary or trinary feedback space provided, e.g., by two numbers like 0 and 1 or three numbers like 0 and 1 and 2 or by colors like green and red or by three colors like green and yellow and red.

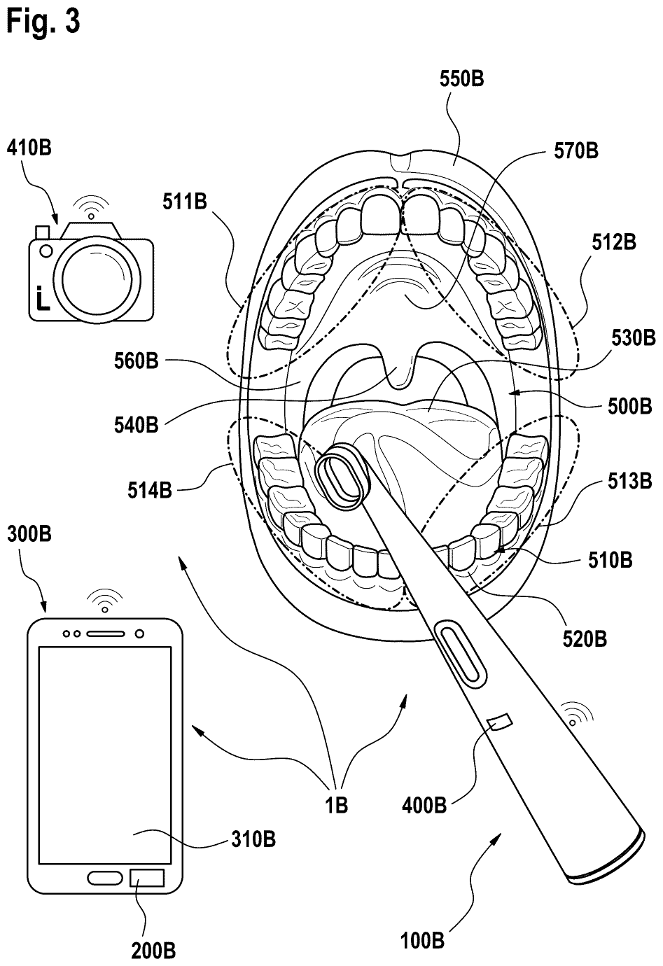

As has already been explained and as will be further explained with reference to FIG. 3, the oral scanner system 1 may additionally comprise at least one position sensor that is coupled or connected with the processor 200 so that the processor 200 receives in operation signals from the position sensor that deliver position sensor data from which the processor 200 can determine a discrete position or location at which the oral scanner is currently performing a scanning procedure or has been performing a scanning procedure at a given time instant in the oral cavity. As has been defined before, a discrete position or a discrete location means a segment of the at least portion of the oral cavity that is being scanned so that the plurality of segments in a gap-free manner and without overlap cover this portion of the oral cavity that is being scanned. Time data relating to an absolute or relative time at which the data was acquired may be part of the position sensor data and also of the previously mentioned oral health sensor data. The determination of a discrete position or location allows that that the processor 200 can compute oral health data relating to at least one oral health condition and/or classify the oral health sensor data and/or the oral health data into at least two oral health condition classes in a discrete position or location resolved manner, i.e., for each of the mentioned segments. The oral health sensor data and the position sensor data acquired at essentially the same time instant may be delivered to the processor 200 together due to the design of the oral scanner system or the processor may be structured and/or arranged to assign oral health sensor data and position data having the same time information (‘time stamps’) or best fitting, i.e., closest lying time information (time stamps) to each other. It is noted that while the provision of the position sensor data and/or the oral health sensor data may be done in a live manner, it is also possible that the respective data is stored for a certain time period, preferably together with a time information, and may be transmitted to the processor at a later moment in time, e.g., data may be sent every 10 seconds or after the scanning procedure has stopped or completed. The term “position sensor” shall include embodiments where two distinct position sensors are used that together realize the “position sensor”, e.g., an IMU provided at the oral scanner and a separate camera.

The oral scanner system 1 may comprise a feedback unit 120 to provide user perceptible feedback, specifically feedback that is comprised of or at least includes processed information per segment, i.e., single feedback provided in the form of a color or a single value for each of the segments/discrete positions or locations. E.g., as is exemplary shown in FIG. 1, the oral scanner 100 may comprise a visual feedback unit 121 being part of the feedback unit 120 for visually providing feedback. In FIG. 1, the visual feedback unit 121 comprises four quarter-annulus light areas 1211, 1212, 1213, 1214 that are arranged to form an annulus that may be understood to represent the four quadrants of the dentition. By illuminating the light areas 1211, 1212, 1213, 1214 with light of different colors and/or with different intensity characteristics, user perceptible feedback can be provided, e.g., live during a scanning procedure so that the user can understand the progress of the scanning procedure in a discrete position or location resolved manner. Additionally or alternatively, the four light areas 1211, 1212, 1213, 1214 may be used to indicate the severeness of an oral health condition during or at the end of the scanning procedure in a discrete position or location resolved manner, e.g., by illuminating the respective light area in a particular color and/or by applying an intensity variation pattern. These are just examples and instead of four light areas, the oral scanner 100 may comprise two or three or five or six or sixteen or thirty-two etc. light areas and/or the oral scanner system 1 may comprise a display to visualize user-perceptible feedback in an even more versatile manner, e.g., may display a value such as a percentage per segment. Reference is made to the previous paragraphs relating to visualization of feedback. The oral scanner 100 may, additionally or alternatively, comprise one or several other feedback elements 122 being part of the feedback unit 120 such as a light ring at the bottom of the oral scanner 100 to communicate that the oral scanner 100 is switched on or that the energy storage requires charging etc., one or several haptic or tactile feedback elements and/or one or several audible feedback elements. Generally, the processor 200 may be coupled or connected with a memory for storing oral health sensor data and/or oral health data and/or scanning progress data and/or condition classification data and/or oral care activity data, where this stored data may be stored in a position-resolved or location-resolved manner and specifically where current and historic stored data may be present, where here “historic” relates to previous scanning procedures or oral care activities. Oral care activity data relates to an oral care activity procedure performed with an oral care device and which data was sent to the processor. All the aspects described with respect to this embodiment indicated in FIG. 1 shall also be understood as being provided for all other embodiments in the present disclosure without repetition of the same text to the extent the individual aspect would not be in conflict with another embodiment.

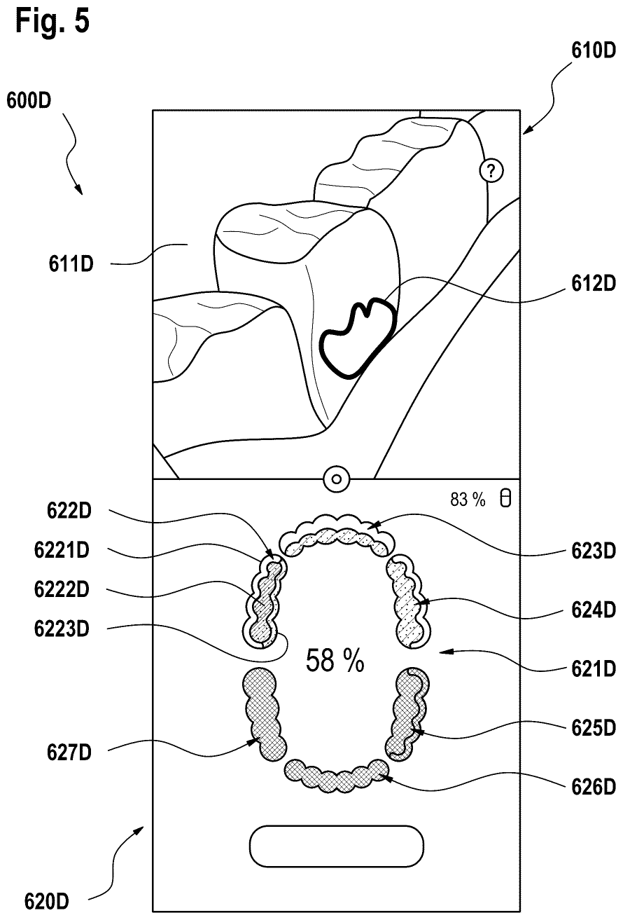

FIG. 2 is a schematic depiction of another example oral scanner system 1A in accordance with the present disclosure. The oral scanner system 1A here comprises an example oral scanner 100A and an example separate device 300A that comprises a processor 200A and a display 310A as part of a feedback unit for visualizing user-perceptible feedback (reference is again made to previous paragraphs providing details on the visualization and to the disclosure further below with reference to FIGS. 5 to 7). The oral scanner 100A may comprise a scanner communicator 140A and the separate device 300A may comprise a separate device communicator 340A so that the oral scanner 100A and the separate device 300A can communicate, i.e., can exchange signals delivering data, in a wireless manner, e.g., via a Bluetooth protocol or an IEEE 820.11 protocol etc. The wireless communication possibility is here and in the following figures indicated by an icon comprising a small circle and three concentric circular segments as is general standard for indicating Wi-Fi connectivity features. This shall not exclude a permanent or temporal additional or alternate wired direct or indirect connection for the exchange of signals or a communication via a further device, e.g., a charger or a router or a cloud computing device etc. The separate device 300A is here schematically indicated to be a mobile phone, even though this shall not be understood as limiting. Reference is made to the possibilities to realize a separate device described in a previous paragraph. In FIG. 2 it is indicated that an oral health sensor 110A is provided in or at the oral scanner 100A for acquisition of oral health sensor data at a head section 102A of the oral scanner 100A. As is indicated, the oral health sensor 110A may comprise a sensor receiver 111A, e.g., an optical sensor such as a camera, and a sensor emitter 112A, such as a light emitter. A preferably detachable attachment 105A is here attached to the head portion 102A that may preferably be realized as a distance attachment. Reference is made to the previous paragraphs relating to attachments of the oral scanner. Instead of a sensor emitter 112A directly being arranged at the head portion 102A, the head portion 102A may comprise an outlet that communicates with the sensor emitter 112A so that the emitted medium can exit the head portion 102A at an intended location and the sensor emitter 112A itself may be disposed elsewhere in the oral scanner 100A. Likewise, an inlet may be provided at the head portion 102A, which inlet may communicate with the sensor receiver 111A so that a medium to be measured can enter the head portion 102A at the intended location and the sensor receiver 111A may be disposed somewhere else in the oral scanner 100A.

Whether the feedback unit is at least partly be provided at the oral scanner and/or at a separate device, the intention of the feedback discussed herein to allow the user to respond to the feedback and thus to optimize the use of the oral scanner system. The use of the oral scanner system is hereby on the one hand focusing on the use of the oral scanner system during a single scanning procedure and on the other hand on a long-term usage of the oral scanner system over various instance of procedures to be performed with the components of the oral scanner system, e.g., comprising the oral scanner and optionally an oral care device for providing an oral care activity.

FIG. 3 is a schematic depiction of an example oral scanner system 1B in accordance with the present disclosure that comprises an oral scanner 100B, a separate device 300B comprising a display 310B as part of a feedback unit and a processor 200B, a position sensor 400B, 410B comprising a first and a second position sensor 400B and 410B, respectively, and is structured and/or arranged to utilize position sensor data outputted by the position sensor 400B, 410B to determine a discrete position or location in an oral cavity 500B at which the oral scanner 100B is currently performing a scanning procedure or has been performing a scanning procedure at a given time instant, where the time instant may be derivable from a time value outputted by the position sensor 400B, 410B together with the relating position sensor data or a clock may be used for absolute time values. As was previously discussed, the position sensor 400B, 410B in this example comprises two position sensors, one disposed in or at the oral scanner 100B and one being separate from the oral scanner 100B.

The oral cavity 500B shown in FIG. 3 comprises, without wanting to be complete, a dentition 510B, gums, 520B, a tongue 530B, a uvula 540B, lips 550B, inner cheeks 560B, and a palate 570B. For the sake of simplicity, only the dentition 510B is further discussed even though all other areas in the oral cavity 500B may be considered as well. For the present exemplary discussion, the dentition 510B is virtually separated into four quadrants 511B, 512B, 513B, 514B that are considered different segments in the oral cavity 500B at which the oral scanner 100B may perform a scanning procedure. It shall be understood that the segments defined in the oral cavity 500B do not need to cover the complete dentition 510B but may only cover a portion thereof, which is then the portion of the oral cavity that is intended to be scanned. The first position sensor 400B is here disposed at or in the oral scanner 100B and may be realized as an accelerometer and/or a gyroscope and/or a magnetometer (generally speaking, as an IMU). Position sensor data and oral health sensor data may be wirelessly transmitted to and received by the processor 200B via a processor communicator as has been already described and the processor 200B may be structured and/or arranged to determine a discrete position or location, i.e., a segments from the list of to be scanned segments at which the oral scanner 100B currently performs a scanning procedure based on the position sensor data or where the oral scanner 100B has been performing a scanning procedure at a given time instant based on the position sensor data that may include timer data. In the present example, the processor 200B may output one of the four dentition quadrants 511B, 512B, 513B, 514B as the scanning segment, i.e., as the currently scanned discrete position or location. The processor 200B may preferably be structured and/or arranged to also output that no scanning currently occurs in any of the discrete positions or locations that were defined. E.g., in case the oral scanner 100B is being moved outside of the oral cavity 500B or across the tongue 530B, then the processor may output that the scanning procedure occurs at none of the used discrete positions or locations or the processor 200B may explicitly indicate that the oral scanner 200B is outside of the used discrete positions or locations. The processor 200B may be further structured and/or arranged to compute oral health data from the oral health sensor data in a position-resolved or location-resolved, i.e., segment-resolved manner, i.e., by assigning the oral health sensor data and/or the therefrom derived oral health data to the determined discrete position or location (or: segment). Reference is made to the previous paragraphs disclosing details of the discrete position or location determination and how oral health data is assigned to the discrete position or location. In some embodiments, the processor 200B determines an orientation of the oral scanner 100B with respect to Earth's gravity field and determines a discrete position or location (or: segment) by sorting the orientation values into pre-determined discrete position or location (or: segment) buckets as is known in the art.

In addition or alternatively, the second position sensor 410B may be utilized which in this example is a separate camera that takes images from the outside of or within the oral cavity 500B, where images are understood to be position sensor data delivered by the camera 410B. Either based on the pictures alone and/or based on data fusion with the position sensor data from the first position sensor 400B a discrete position or location (or: segment) in the oral cavity 500B may be determined by the processor 200B, where here the discrete position or location relates to one of the indicated dentition quadrants 511B, 512B, 513B, 514B. That here an external camera is indicated shall not exclude that alternatively or additionally a camera is used as position sensor that is disposed at a head portion or at a handle portion of the oral scanner 100B so that images from inside of the oral cavity 500B or images from the face of the user can be taken, respectively, to support in the determination of the discrete position or location (or: segment). In accordance with some aspects of the whole description, a camera serving as oral health sensor may additionally be utilized as position sensor-see, e.g., the reference made to EP 2 189 198 B1 in a previous paragraph. A scanning procedure performed with an oral health sensor comprising an optical sensor such as a camera is called an optical scanning procedure.

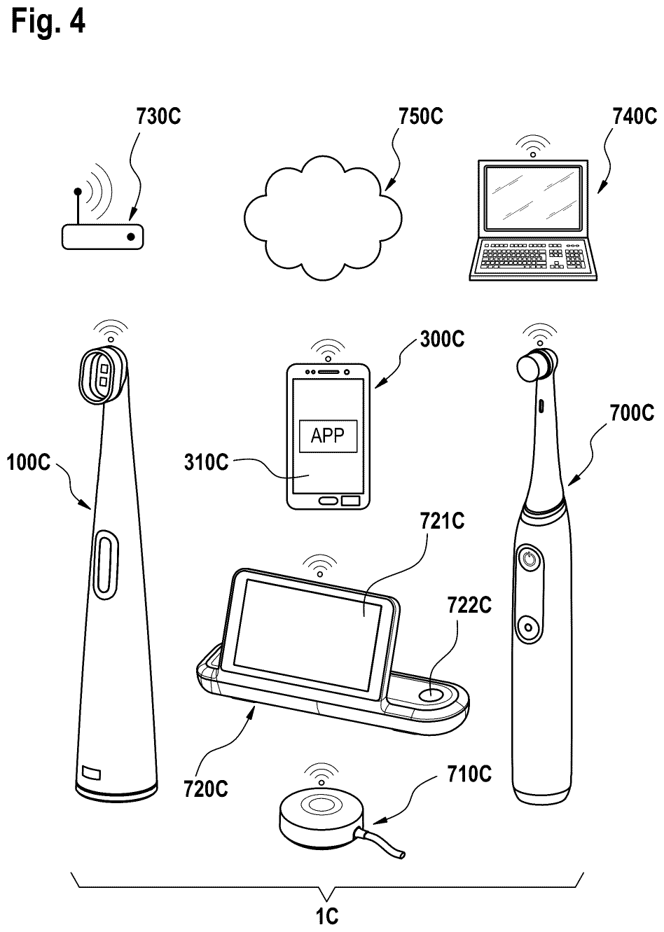

FIG. 4 is a schematic depiction of an example oral scanner system 1C in accordance with the present disclosure specifically comprising an oral care device 700C, even though several aspects of the oral scanner system 1C are independent from the presence of the oral care device 700C. The oral scanner system 1C may comprise or interact with an oral scanner 100C, a separate device 300C comprising a display 310C, the mentioned oral care device 700C that here is exemplified as an electric toothbrush, a charger 710C, a base station 720C comprising a display 721C and a charger 722C, a router 730C, a computer 740C and a cloud server or cloud computing device 750C. The various components of the oral care system 1C may preferably all be structured and/or arranged for wireless communication as is indicated with the previously mentioned icon. It shall be understood that the here shown components of the oral scanner system 1C are an optional assembly. E.g., the oral scanner system 1C may comprise only one charger or no charger at all or may indeed comprise two chargers, one for the oral scanner 100C and one for the oral care device 700C and potentially a further charger for the separate device 300C. As was already explained in a previous paragraph, a processor of the oral scanner system 1C may be realized as a distributed processor and a first processor sub-unit may be disposed in the oral scanner 100C and a second processor sub-unit may be provided by the cloud computing device 750C or a first processor sub-unit may be provided by the separate device 300C and a second processor sub-unit may be provided by the computer 740C. Reference is made to a previous discussion on how the oral care device 700C may be incorporated into the oral scanner system 1C and that at least one operational setting of the oral care device 700C may be selected based on control data determined by the processor and/or that the oral care device 700C may be structured and/or arranged to send oral care activity data relating to at least one oral care activity performed with the oral care device 700C to the processor where it may be used to adapt a next scanning procedure. Data from one component may be sent directly to another component, e.g., from the oral care device 700C to the oral scanner 100C, or may be sent indirectly, e.g., from the oral care device 700C to the cloud server 750C, where it may be stored in a memory, and then, e.g., on demand, from the cloud server 750C to the processor which may be located in or at the separate device 300C and/or in or at the oral scanner 100C. The mentioned memory may be a memory located in any of the mentioned components or may be a distributed memory.

FIG. 5 is a depiction of an example feedback screen 600D as may be visualized on a display of an oral scanner system. The term feedback screen here refers to a visualization of feedback to a user by means of a display using a particular feedback concept within a continuous guidance provided to the user by the oral scanner system. Feedback screens are preferably used to assist the user in performing the task of using the oral scanner system by means of a continued or guided human-machine interaction process, which shall not exclude that a feedback screen in addition also visualizes information such as the current time etc. It should be understood that individual aspects of a feedback screen shown here shall not be understood as necessarily been disclosed together, but that the different feedback screen aspect may be assembled in an arbitrary manner and that the examples provided in the images are exemplary only. In FIG. 5, the feedback screen 600D comprises a first portion 610D and a second portion 620D. On the first portion, a live image or conserve image 611D from a camera on a head portion of an oral scanner of the oral scanner system is shown. The camera may be comprised by an oral health sensor. The live image may comprise unprocessed or processed image data relating to an oral health condition, e.g., to plaque image data visible as red fluorescence light. A processor may be structured and/or arranged to analyze the image data and may determine a borderline within the image or an image portion in which the relevant oral health sensor data is located and a respective indication 612D may be overlaid onto the live image 611D and be visualized as well as part of the live or conserve image. The indication 612D is shown in FIG. 5, which indication is overlaid onto the visualized image data 611D and shall provide a visible reference of the area of the tooth that is visible on the image covered by plaque. It is stated here that the indication 612D is derived from camera data, specifically from camera data imaging fluorescence light, where the indication 612D shows the area on a currently scanned tooth (live image) or on a conserve image (e.g., as it shows the tooth having the most sever issues) where a scanned oral health issue such as plaque was found. While the indication 612D is the result of processing optical oral health data captured by a camera, the indication 612D itself has no meaning without overlaying it onto an image of the respective portion of the oral cavity to which it relates. Only further processing such as computing a normalized area of plaque in relation to the total tooth area within a given segment (discrete position or location) would allow to display an easily understandable single value to the user per segment.

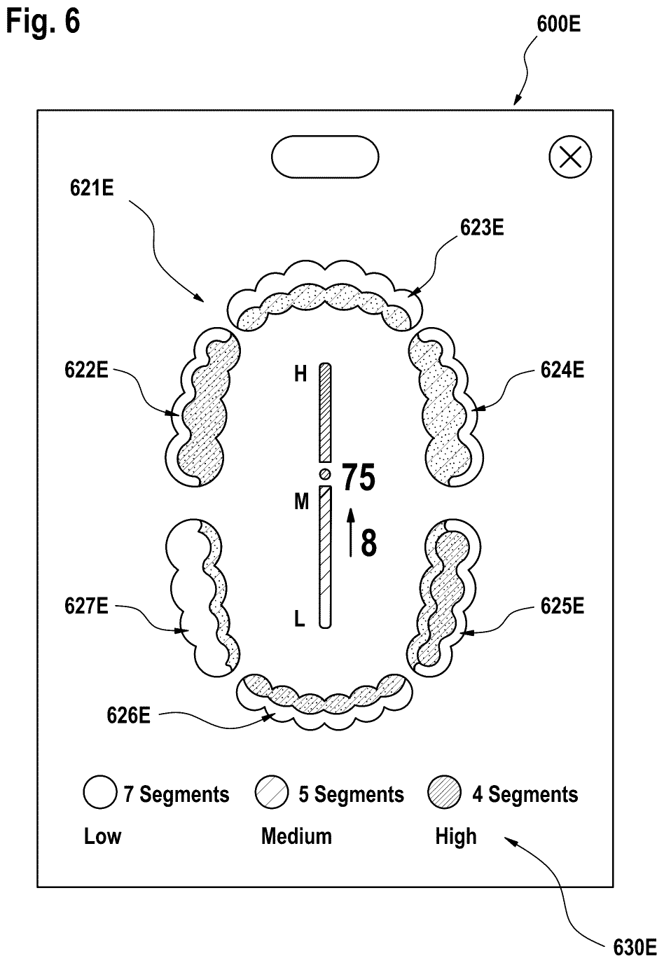

In the shown example, the second portion 620D of the feedback screen 600D comprises an abstract visualization of a human dentition 621D. In the shown example, the abstract visualization of the human dentition 621D comprises six segments (reflecting the scanned segments or discrete positions/locations) 622D, 623D, 624D, 625D, 626D and 627D generally arranged with a distance between two neighboring segments in an oval-like arrangement. Each of the segments 622D, 623D, 624D, 625D, 626D and 627D comprises a plurality of overlapping circles or bubbles, which is understood to be a non-limiting example of a visualization possibility. The top three segments 622D, 623D, 624D shall indicate the teeth of the maxilla and the lower three segments 625D, 626D, 627D shall indicate the teeth of the mandible. The top segment 623D and the bottom segment 626D shall represent locations in the dentition relating to the upper and the lower front teeth, respectively, the left-hand segments 622D and 627D shall represent locations in the dentition relating to the upper and lower left molars, respectively, and the right-hand segments 624D and 625D shall represent locations in the dentition relating to the upper and lower right molars, respectively. With reference to segment 622D (and also with respect to segment 625E of FIG. 6 as a further example) it is indicated that a shown abstract segment may be visually separated into two or three or even more subdivisions (segments) that then may relate to different discrete positions or locations of the dentition. These subdivisions may be used to visually distinguish, e.g., different teeth or groups of teeth relating to a higher order segment or different tooth surfaces or groups of tooth surfaces relating to the higher order segment. Segment 622D (and also segment 625E in FIG. 6) is separated into three areas 6221D, 6222D, 6223D, where the side areas 6221D and 6223D shall represent the buccal and lingual surfaces of the molar teeth of the segment 622D, respectively, and the center area 6222D shall represent the occlusal or biting surface of the molar teeth of the segment 622D. Such a portion of a feedback screen, whether as just one portion of the overall feedback screen or as essentially the only portion of the feedback screen may be used to provide live or summary feedback to the user. FIG. 5 shows a feedback screen as may be seen by a user during a live scanning procedure. The segments 622D, 623D, 624D, 625D, 626D and 627D may be used to indicate a position-resolved or location-resolved scanning procedure progress and/or a severity of an oral health condition, e.g., the total or normalized tooth area within a segment on which plaque or the like was determined. Again, it is noted that the segments or the subdivisions of the segments shown on the feedback screen relate to discrete positions or locations in the oral cavity. As was already discussed in a previous paragraph, the scanning procedure progress may be visualized by first showing all segments and all segment subdivisions if such are used in a base or start color (e.g., dark blue) or a start pattern or the like and to then gradually or step-wise change the color or pattern or the like towards a different color or pattern, e.g., towards lighter blue and finally white to indicate a scanning procedure progress for the respective segment, i.e., for the respective discrete position or location. While it may be preferred to have more than two colors or patterns used per segment or subdivision of a pattern to indicate a level of scanning procedure progress or severity of an oral health condition, it shall not be excluded that only two colors or patterns etc. are used. In FIG. 5, a shading of different strength is used instead of color. The severity of the detected oral health condition is determined based on the discrete position or location resolved oral health (sensor) data and may be visualized by adding a pattern of different strength into the color. In FIG. 5 additional dots are used to indicate the severity of the oral health condition.

FIG. 6 is a depiction of an example feedback screen 600E as may be visualized on a display of an oral scanner system. The feedback screen 600E comprises essentially the same abstract visualization of the dentition 621E as was explained with respect to FIG. 5 and reference is made to the respective description. Abstract segments 622E, 623E, 624E, 625E, 626E, and 627E are shown. Feedback screen 600E can be understood as a summary screen on which the severeness of the detected oral health condition, e.g., plaque, is indicated by different colors or patterns or the like in a discrete position or location resolved manner (in FIG. 6 a shading of different strength is used). In this embodiment, the basic feedback concepts as discussed with respect to FIG. 5 are used for indicating the live status of the oral health condition in each of the segments or the final status of the oral health condition at the end of the scanning procedure instead of the live or final scanning progress discussed for FIG. 5. Additional patterns or structures may be applied to indicate additional feedback, e.g., the presence of another oral health condition such as tartar (i.e., old plaque), where the strength of the pattern or the number of additional structures may be indicative of the severeness of the additional oral health condition. In FIG. 6 additional dots are shown. Further, feedback screen 600E comprises a visualization of a temporal change relating to the severeness of at least one oral health condition, e.g., plaque. Such visualized feedback may indicate in an appropriate manner the severeness of the oral health condition as determined in the recent scanning procedure and a change indicator that provides feedback on the change of the severeness in comparison to at least one previous scanning procedure. The bar indicator with the temporal change arrow as shown in FIG. 6 is just one example of such a visualization of comparison data, i.e., comparison data relating to a comparison of current data with stored historic data. The shown bar indicator comprises a bar indicating the oral health condition, where here the bottom relates to no issue and the top relates to a condition of concern, where a first number, here 75, indicates a normalized oral health condition rating (normalization may here relate to a range between 0 and 100) and a second number, here 8, indicates the temporal change vs. the previous, i.e., historic scanning procedure. A reference guide 630E may be visualized allowing to map the colors or signs or patterns etc. to the severity of an oral health condition, where the severity as indicated in the reference guide 630E may coincide with condition classes into which the oral health data was classified, where in the shown example three condition classes are used, namely “low”, “medium” and “high”. In this example, the information relating to a comparison with historic data is shown as a global indicator for the complete portion of the oral cavity that was scanned. In contrast, it can be imagined that a feedback screen may be shown where the temporal change is indicated in a segment-resolved manner, e.g., as a color of each segment and/or an assigned value may be used to indicate the temporal change to the better or worse for each segment, i.e., for each discrete position or location.

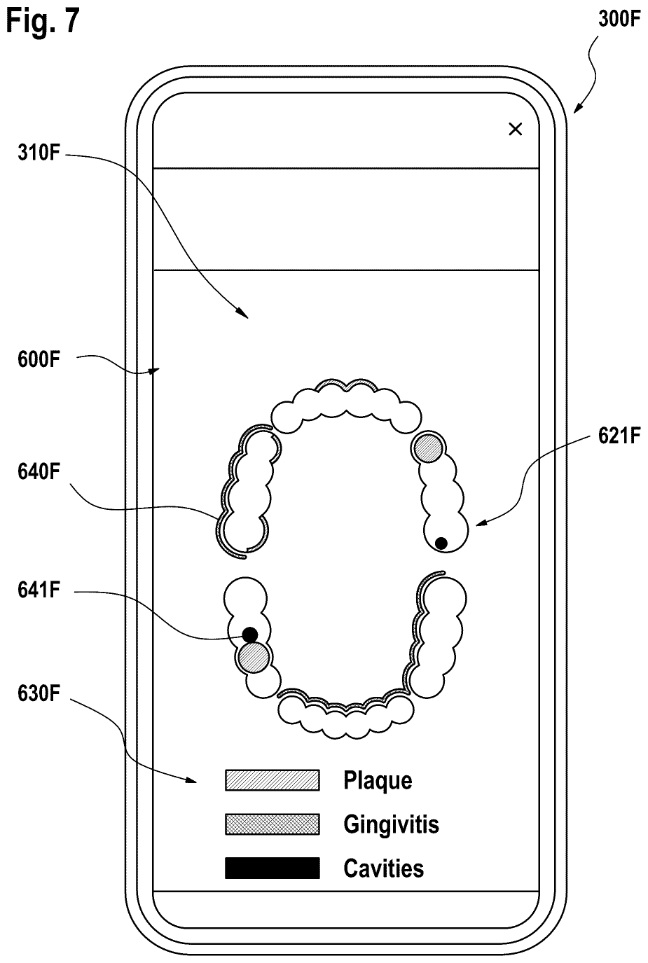

FIG. 7 is a depiction of an example separate device 300F that is part of an oral scanner system and that comprises a display 310F on which an example feedback screen 600F is visualized. Again, an abstract visualization 621F of the dentition is utilized as in FIGS. 5 and 6. In addition to the segments of the dentition, locations 640F relating to segments of the gums are indicated where an oral health condition of a certain severeness was detected (i.e., where the analysis of the oral health sensor data led to an oral health condition above a threshold value), e.g., where an inflammation of the gums was detected based on an analysis of, e.g., image data created by a camera as sensor receiver of an oral health sensor. Feedback screen 600F provides one example of a visualization to provide feedback about various oral health conditions classified into different condition classes. A reference guide 630F may be visualized allowing to map the colors or signs or patterns etc. to the type of oral health condition and its classification. Visualized indicia 640F, 641F may be overlaid onto the abstract visualization 621F of the dentition to provide feedback about further oral health conditions, e.g., cavities or the like. The size of such an indicium 641F may relate to a severeness and thus to a condition class. As is indicated in FIG. 7, the individual discrete positions or locations may be shown on an even more resolved manner, e.g., resolved on the level of an individual tooth. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. An oral scanner system comprising:
an oral scanner comprising an oral health sensor structured and/or arranged for acquiring oral health sensor data from at least a portion of a dentition of a subject during a scanning procedure relating to at least one oral health condition; and
a position detector structured and/or arranged for acquiring position sensor data during the scanning procedure;
a processor structured and/or arranged
to receive the oral health sensor data and the position sensor data,
to process the position sensor data to determine a discrete position or location from at least two discrete positions or locations of the at least portion of the dentition at which the oral scanner is currently performing the scanning procedure or has been performing the scanning procedure at a given time instant,
to assign the oral health sensor data and/or oral health data determined by the processor based on the oral health sensor data to one discrete position or location of the at least two discrete positions or locations to create discretely position-resolved or location-resolved oral health sensor data and/or oral health data,
to compare the discretely position-resolved or location-resolved oral health sensor data and/or oral health data with historic discretely position-resolved or location-resolved oral health sensor data and/or historic oral health data, respectively, being stored in a memory of the oral scanner system and having been determined in at least one previous scanning procedure, and
to generate comparison data, preferably discretely position-resolved or location-resolved comparison data, and
to control a display unit to provide a visualization of the discretely position-resolved or location-resolved oral health sensor data and/or the oral health data at least after the scanning procedure is complete and/or to provide a visualization of the comparison data, preferably the discretely position-resolved or location-resolved comparison data.

2. The oral scanner system in accordance with claim 1, wherein the processor is structured and/or arranged to provide a position-resolved visualization of the comparison data.

3. The oral scanner system in accordance with claim 2, wherein the position sensor comprises at least one from the list comprising an accelerometer and a gyroscope, preferably wherein the accelerometer or gyroscope is realized as a MEMS sensor.

4. The oral scanner system in accordance with claim 3, wherein the processor is structured and/or arranged
to assign the oral health sensor data and/or the oral health data to the determined discrete position or location at which the oral scanner is currently performing the scanning procedure so that the oral health sensor data and/or the oral health data becomes position resolved or location resolved, and
preferably to compare the position resolved or location resolved oral health sensor data and/or the position resolved or location resolved oral health data with historic position resolved or location resolved oral health sensor data and/or historic position resolved or location resolved oral health data being stored in a memory and having been determined in at least one previous scanning procedure to generate position resolved or location resolved comparison data.

5. The oral scanner system in accordance with claim 4, wherein the processor is structured and/or arranged to control the feedback unit to provide user-perceptible feedback about the position resolved or location resolved oral health sensor data and/or the position resolved or location resolved oral health data.

6. The oral scanner system in accordance with claim 5, wherein the processor is structured and/or arranged to control the feedback unit to provide user-perceptible feedback about the position resolved or location resolved comparison data, preferably visually perceptible feedback.

7. The oral scanner system in accordance with claim 6, wherein the processor is structured and/or arranged
to either process the oral health sensor data to determine oral health data relating to the at least one oral health condition, to classify the oral health data with respect to at least two condition classes relating to the at least one oral health condition and to determine one condition class from the at least two condition classes to which the oral health data belongs, or
to classify the oral health sensor data with respect to at least two condition classes relating to the at least one oral health condition and to determine one condition class from the at least two condition classes to which the oral health sensor data belongs.

8. The oral scanner system in accordance with claim 7, wherein the processor is structured and/or arranged to classify the position resolved or location resolved oral health sensor data and/or the position resolved or location resolved oral health data with respect to at least two condition classes relating to the at least one oral health condition, preferably based on classification requirements that are different for at least two positions or locations of the at least two positions or locations of the at least portion of the oral cavity.

9. The oral scanner system in accordance with claim 8, wherein historic classification data is stored in the memory, preferably wherein the historic classification data includes at least one historic condition class determined in at least one previous scanning procedure, further preferably wherein the historic classification data includes at least two historic position-resolved or location-resolved condition classes determined in at least one previous scanning procedure.

10. The oral scanner system in accordance with claim 1, wherein the oral health sensor comprises an optical sensor, preferably an M time N array of light sensitive sensor elements.

11. The oral scanner system in accordance with claim 1, wherein the oral health sensor is a camera for acquiring image data and wherein the processor is structured and/or arranged to process the image data to derive the oral health data as a set of numerical values in a position resolved or location resolved manner.

12. The oral scanner system in accordance with claim 1, wherein the display unit is realized as or by a separate device such as the separate device is a computer, a notebook, a laptop, a tablet, a smartphone, or a smart watch.

13. The oral scanner system in accordance with claim 12, wherein the oral scanner comprises a communicator and the display unit comprises a communicator and the communicators are structured and/or arranged for at least a unidirectional wireless communication from the oral scanner to the display unit.

14. The oral scanner system in accordance with claim 1, wherein the feedback unit is structured and arranged to visualize a depiction of the at least portion of the oral cavity, preferably comprising at least a visualization of the dentition virtually separated into at least two discrete segments representing the at least two positions or locations, and wherein at least the oral health sensor data and/or the oral health data, preferably for at least two of the at least two positions or locations, and the comparison data is additionally visualized, preferably wherein at least the oral health sensor data and/or oral health data is visually overlaid onto the depiction of the dentition or where the depiction of the dentition is amended based on at least the oral health sensor data and/or the oral health data.
